(19) United States
(12) Patent Application Publication
(10) Pub. No.: US 2024/0177505 A1
(43) Pub. Date: May 30, 2024

(54) Method and apparatus for determining a signal composition of signal series from an image series

(71) Applicant: Carl Zeiss Microscopy GmbH, Jena (DE)

(72) Inventors: Manuel AMTHOR, Jena (DE); Daniel HAASE, Zoellnitz (DE); Ralf WOLLESCHENSKY, Jena (DE)

(21) Appl. No.: 18/519,282

(22) Filed: Nov. 27, 2023

(30) Foreign Application Priority Data
Nov. 28, 2022 (DE) 1020221314510

(51) Int. Cl. G06V 20/69; G06T 7/11; G06T 7/194; G06T 7/90; G06V 10/762; G06V 10/774; G06V 10/82

(52) U.S. Cl. CPC G06V 20/698; G06T 7/11; G06T 7/194; G06T 7/90; G06V 10/762; G06V 10/774; G06V 10/82; G06V 20/695; G06T 2207/10024; G06T 2207/10056; G06T 2207/20076; G06T 2207/20084; G06T 2207/20224; G06T 2207/30024; G06T 2207/30204

(57) ABSTRACT

Method for determining a signal composition of signal series of an image series with an analyte data evaluation system, wherein the image series is generated by marking analytes with markers in a plurality of coloring rounds and detecting the markers with a camera, the camera acquires an image of the image series in each coloring round, the markers being selected in such a way that the signal series of analytes in an image area across the image series comprise colored and uncolored signals, and that the signal series of different analyte types each have a specific sequence of colored signals and uncolored signals, and the different analyte types can be identified based on the specific sequences, comprising: receiving the signal series; importing a codebook, wherein the codebook comprises a target series for all signal components and the target series comprise analyte target series.

The present invention relates to a method and apparatus for determining a signal composition of signal series of an image series, and to a method and apparatus for training a machine learning system with a processing model that is trained to determine a signal composition of signal series of image areas from an image series.

EP 2 992 115 B1 describes a method for identifying analytes by coloring the analytes to be identified with markers in a plurality of coloring rounds. The markers consist of oligonucleotides and dyes coupled thereto, which are generally fluorescent dyes. The oligonucleotides are specific for certain sections of the analytes to be identified. The individual oligonucleotides of the markers, however, are not unique to the particular analytes. But due to the plurality of coloring rounds, it is possible to clearly identify the analytes because a plurality of different markers can be assigned to a specific oligonucleotide after the plurality of coloring rounds has been completed, making the assigned markers unique to the respective analytes.

This method can be used to detect a wide variety of analytes in vitro, for example in a cell, with the help of a fluorescence microscope. The analytes can be an RNA, in particular an mRNA or a tRNA. The analytes can also be a section of a DNA.

A sample often comprises a plurality of analytes that can be identified in parallel with the coloring rounds described above, even if they are different analytes. The more analytes there are in the sample, the greater the number of markers to be detected in the respective coloring rounds. In the case of an automatic capture and evaluation of the corresponding image signals, the image signals of all markers in the sample must be captured and likewise distinguished from image signals in the sample not caused by markers coupled to analytes.

WO 2020/254519 A1 and WO 2021/255244 A1 describe a further method that can be used, among other things, to identify not only analytes, but proteins as well. In this method, probes specific to the respective analytes are first coupled to said analytes. The probes have oligonucleotide residues that do not hybridize with the analytes. Decoding oligonucleotides are hybridized with the free oligonucleotide residues and have a supernatant to the free residues. Using a dye, marker molecules, or markers for short, are hybridized at the supernatants. In this method as well, a series of image signals is generated on the corresponding analytes in a plurality of coloring rounds, which then provide information about the analyte present in each case. Other methods are known as well, however, in which the markers bind directly to the free oligonucleotide residues.

After the acquisition of the images, the signal series of the image signals that were acquired during the coloring rounds are subjected to an analysis in which the signal series are assigned to the analyte types. It has been shown that the analysis of the signal series does not always provide clear results.

### SUMMARY OF THE INVENTION

The invention is based on the task of providing a method with which a signal composition of signal series of an image series can likewise be determined for signal series that are composed of signal series from a plurality of analytes.

A further object of the invention is to provide a method that makes it possible to train a machine learning system to determine a signal composition of signal series of an image series even for signal series composed of signal series from a plurality of analytes.

One aspect of the invention relates to a method for training a machine learning system with a processing model. The processing model is trained to determine a signal composition of signal series from image areas of an image series. The image series is generated by marking analytes with markers in a plurality of coloring rounds and detecting the markers with a camera. The camera captures an image of the image series in each coloring round. The markers are selected in such a way that the signal series of analytes in an image area across the image series include colored signals and uncolored signals. The colored and uncolored signals of the analyte signal series have at least one particular ratio of one of the colored and/or uncolored signals of the respective signal series to at least one other of the colored and/or uncolored signals of the respective signal series, or the analyte signal series have a characteristic signature comprising the at least one particular ratio. The method comprises a step of providing an annotated data set, the annotated data set comprising input signal series for various signal components to be identified, and corresponding target outputs. The signal components comprise at least one signal component for each analyte type to be identified. The analyte signal series have a specific order of colored and uncolored signals, based on which an analyte type can be assigned to the signal series. The method further comprises a step of optimizing an objective function by adjusting the model parameters of the processing model, wherein the objective function is calculated based on a result output from the processing model and the target output.

According to the present invention, an analyte is an entity the presence or absence of which is to be specifically detected in a sample and which, if present, is to be coded. This may be any type of entity, including a protein, a polypeptide, a protein molecule, or a nucleic acid molecule (e.g., RNA, PNA, or DNA), also called a transcript. The analyte provides at least one site for a specific binding with analyte-specific probes. An analyte in accordance with the invention may comprise a complex of items, e.g., at least two individual nucleic acid molecules, protein molecules, or peptide molecules. In one embodiment of the disclosure, an analyte excludes a chromosome. In another embodiment of the disclosure, an analyte excludes DNA. In some embodiments, an analyte can be a coding sequence, a structural nucleotide sequence, or a structural nucleic acid molecule that refers to a nucleotide sequence that is translated into a polypeptide, typically via mRNA, when under the control of appropriate regulatory sequences. The boundaries of the coding sequence are determined by a translation start codon at the 5′ terminus and a translation stop codon at the 3′ terminus. A coding sequence can include, but is not limited to, genomic DNA, cDNA, EST, and recombinant nucleotide sequences. Depending on what analyte type is to be identified, such methods are referred to as spatial transcriptomics or also multiomics.

In the following, the term image signal is intended to refer either to a value of a pixel of the image for a specific color of a predetermined color channel, or the image signal comprises values of different basic colors of a color space of a colored image.

In the following, the term signal series is intended to indicate that the signal series comprises the image signals of image areas from the image series, whereby the image areas from the different images of the image series are registered to each other. Accordingly, the image areas capture image signals from the same location in the sample in all of the images of the image series. The signal series of an image area comprises the image signals of the images of the image series of the respective image area.

In the following, the term signal composition is intended to indicate that a signal composition comprises a signal portion for a plurality of possible signal components or signal components to be identified. The signal components can, for example, be signal components of different analyte types, but also signal components of background signals. A signal portion here can be an absolute signal portion, a relative signal portion, or also just a binary signal portion, i.e., the signal composition indicates only which of the possible signal components contribute to a signal series.

According to the present invention, the spectral ranges that each comprise one color of a marker are also called color channels. The images separated into the color channels are mono-chromatic images and for every individual pixel, contain the above-described image signal of the pixel in the color of the color channel as a value or measured value.

The inventors have realized that signal series of image areas that capture image signals from analytes each have at least one particular ratio of colored and/or uncolored signals from the respective signal series across the signal series. Accordingly, signal series originating from analytes comprise a characteristic signature with the at least one particular ratio of the colored and/or uncolored signals of the signal series. Furthermore, for each of the analyte types to be identified, the analyte signal series have a particular sequence of colored and uncolored signals, based on which the analyte signal series can be assigned to an analyte type. Due to the fact that, according to the method for training a machine learning system, a processing model with signal series comprising colored and uncolored signals with the particular ratio or the characteristic signature, respectively, and the specific sequence of colored and uncolored signals, is trained to identify an analyte type, it is possible to provide a very effective, fast, and well-controlled method for training a machine learning system with a processing model that assigns signal portions from signal components to signal series from image areas from an image series. A machine learning system trained in this manner can analyze the data of an image series with marked analytes in a very efficient manner and also reliably assign signal series with signal portions from a plurality of signal components to said signal series.

Preferably, the annotated data set further comprises input signal series from background image areas, wherein the background image areas are image areas from the image series in which no signals from analytes are captured, and the target output for the background image areas forms at least one signal component of its own in the set of signal components.

The fact that a signal from a background image area is included as a separate signal component in the analysis of the signal components and is taken into account during training further improves the detection and assignment of signal portions to the signal components.

Preferably, the processing model is a classification model, and the result output is a signal component of the input signal series. Alternatively, the result output is a probability distribution, which respectively indicates the probability of belonging to one of the signal components, and the objective function detects a difference between the result output and the target output.

Because the processing model is trained as a classification model for outputting the signal components, the signal portion can be easily assigned to the respective signal component on the basis of an output of the processing model; no further matching is necessary. If the classification model is trained to output a probability distribution, the result can also be used to directly determine how confident the processing model is in assigning the signal component, which makes it possible for the user to check the corresponding assignment in the event of a doubtful assignment, which is particularly desirable. Accordingly, the present invention provides a method for training a machine learning system that can be used to easily train a machine learning system to identify signal portions of signal components from a signal series.

Preferably, an objective function is optimized in a plurality of rounds, wherein, in some of the rounds, the sequence of the colored and uncolored signals of one of the input signal series is changed in such a way that the changed sequence corresponds to a sequence of one other of the analyte types to be identified, and the target output corresponding to the changed sequence is used to optimize the objective function.

By appropriately changing the sequence of the colored and uncolored signals of one of the input signal series to result in a sequence of one other of the analyte types to be identified, an input signal series can be designed, which is used to train the network to identify an analyte type for which no input signal series is available for training purposes.

Preferably, the objective function is a classification loss and the result output for each of the entries is a value between 0 and 1 indicating a probability that the respective signal series belongs to the respective signal component.

The classification loss can, for example, be a cross entropy loss, a hinge loss, a logistic loss, or a Kulback-Leibler loss.

By using a classification loss during training, a probability output can be generated in a particularly simple way.

Preferably, the target output is a target bit series, with the target output comprising a true bit for each colored signal in the input signal series and a false bit for each uncolored signal.

Due to the fact that the target output is a target bit series, a result output of the processing model can be matched particularly easily; in addition, the target bit series require only a little memory, so that the annotated data set can be made available in such a way that it uses as little memory as possible.

Preferably, the result output is a probability distribution in which each image signal of the input signal series is assigned a probability as to whether or not the image signal is a colored signal. The objective function detects a difference between the result output and the target output.

Since the result output is a probability distribution, a user checking the output results can easily determine whether the processing model has detected the respective colored signals with a high degree of certainty. Thus, the method allows for a particularly easy interpretation of the output results.

Preferably, the entries of the result outputs are each a value between 0 and 1, indicating a probability that the respective image signal of the signal series is a colored signal.

The objective function can, for example, be an L1 norm, an L2 norm, a cross entropy loss, a hinge loss, a logistic loss, or a Kullback-Leibler loss.

Preferably, the processing model is a fully convolutional network that was trained as a classification model with fully connected layers by means of signal series of individual image areas, wherein after the training, the classification model is transformed, through replacement of the fully connected layers with convolutional layers, into the fully convolutional network. The fully convolutional network processes the signal series of all image areas from the image series simultaneously. According to one alternative, the fully convolutional network can directly be trained as such.

By training the fully convolutional network as a classification model with fully connected layers, it is possible to save computational power during the training by using signal series of individual image areas, since the entire image series does not always have to be inferred.

Preferably, a calculation of the objective function comprises a calculation of a candidate group of candidate objective functions for each input signal series of analytes. For each of the candidate objective functions, another signal of the colored signals in the input signal series is not taken into account when calculating the candidate objective function, for example, by setting it to zero or replacing it with an uncolored signal. When calculating the candidate objective function for input signal series of a background image area, one or more colored signals contained in input signal series of background image areas are not taken into account when calculating the candidate objective functions by not including the corresponding colored signals in the calculation or replacing them with uncolored signals. After a group of candidates has been calculated, an objective function of choice is selected from the group of candidates. The objective function of choice is the candidate objective function that has either a second largest or a third largest or a fourth largest difference between the target bit series and the result bit series, preferably a second largest difference.

According to the present method, the target bit series are selected before the image series is recorded in such a way that the various analyte types to be identified have a certain Hamming distance. The Hamming distance is understood to be a measure of the difference between character strings, in this case bit series, for example. The Hamming distance of two blocks with the same length is the number at different places.

The Hamming distance is selected in such a way that the analyte type to be identified can still be detected even with an error of one bit, for example. By determining the objective function of choice as described here, the processing model can thus be taught to reliably recognize even incorrectly captured signal series.

Preferably, the processing model is an embedding model that determines an embedding in an embedding space for embedding inputs. The embedding inputs comprise the input signal series and the target outputs. The result outputs comprise the embeddings of the input signal series. Target embeddings comprise the embeddings of the target outputs. Optimizing the objective function minimizes the difference between the embeddings of embedding inputs of the same signal component while simultaneously maximizing the difference between the embeddings of embedding inputs of different signal components.

By choosing the objective function in such a way that the target bit series of an analyte type and the corresponding input signal series are embedded in the embedding space in such a way that their difference is minimized, it is easily possible to assign the target bit series to the captured signal series. In addition, a comparison of the target bit series with the captured signal series is performed directly in the model, which significantly increases the processing speed, since the method can be executed directly on, for example, a graphics card or a special acceleration card for machine learning, such as a tensor processor or an application-specific chip.

Preferably, the target bit series and the input signal series are input into different processing paths of an input layer of the embedding model.

By inputting the target bit series and the input signal series into different processing paths of an input layer of the embedding model, the embedding model comprises different model parameters for the target bit series and the input signal series, which is why they can be appropriately embedded in the embedding space. Therefore, by using different processing paths, a distance in the embedding space is reduced and it is easier to distinguish the analyte types from each other.

Preferably, the optimization of an objective function comprises a plurality of rounds, with a randomization of the input signal series being performed in some of the rounds. The randomization comprises: Swapping a sequence of the image signals of the input signal series, as well as correspondingly swapping the corresponding entries of the target output and a random selection of a first number of colored signals and a second number of uncolored signals from the set of input signal series and the creation of the corresponding target output.

According to the prior art, before an experiment relating to the spatial determination of analytes, target bit series are defined that can be used to identify different analyte types. Depending on the analyte type comprised in the respective samples, different sets of target bit series are used. By randomizing the input signal series, the processing model can be trained to recognize analyte signal series independently of the target bit series newly defined for each new experiment. Thus, a model can be trained to recognize signal series from analytes and then applied to completely different sets of target bit series.

Preferably, an objective function is optimized in a plurality of rounds, with an augmentation of the input signal series occurring in some of the rounds. The augmentation may include, for example, one or more of the following: replacing at least a single one of the colored signals of the input signal series with an uncolored signal, with the uncolored signal being generated either by lowering the colored signal or by replacing the colored signal with an image signal from the vicinity of the image area of the input signal series, from another round of coloring, or from another location in the sample; randomly adding noise to some of the image signals of the image series, for example, the image signals of an input signal series, of one of the images of the image series, or of all images of the image series; shifting and/or rotating the images of the image series with respect to each other, for example by less than two pixels or less than or equal to one pixel, for example half a pixel; replacing a single one of the uncolored signals of the input signal series with a colored signal; shifting the image signals of at least one of the images of the image series by a constant value; or shifting the image signals of the input signal series by a constant value.

The augmentation of the input signal series can make a training of the processing model more robust.

Preferably, the input signal series are transformed into transformed input signal series by means of a transformation and the transformed input signal series are input into the processing model. Potential transformations may be one or more of the following: a main component analysis, a main axis transformation, a singular value decomposition, and/or a normalization, with the normalization comprising a normalization of the image signals via an image or a normalization of the image signals via a signal series, or both.

By inputting transformed signal series into the processing model, for example, certain background components that are extracted by means of the main axis transformation or singular value decomposition can be easily assigned or detected in the processing model, which significantly improves the training of the processing model. Preferably, for example, only a subset of the components of the transformed signal series is input into the processing model.

It turns out that, when performing a suitable transformation, for example with regard to the main component analysis, a first component in the transformed data produces a very large variance but does not contribute to the separation of the analytes. This first component can also be interpreted as the brightness; based on this component, either the other components can be normalized, or the first component can be left out directly. By leaving out the first main component, a background correction is not necessary, which saves time in the further analysis.

Preferably, the annotated data set is generated with at least one of the following: simulating signals of the various markers using a representative background image and a known point spread function of the microscope; generating the annotated data set using a generative model trained on comparable data, acquiring reference images comprising at least one background image and, for each of the background images, at least one image for each of the analyte types, in which analytes of the respective analyte type are marked; performing a classical method for the spatial identification of analytes; acquiring a representative background image and subtracting the image signals of the representative background image pixel by pixel from the image signals of the image series on which the annotated data set is based, prior to providing the annotated data set, so that the annotated data set comprises only background-corrected signal series.

For example, the generative model used may be one of the following models: an active appearance model (AAMs), a generative adversarial network (GANs), a variational autoencoder (VAEs), an auto-regressive model, or a diffusion model.

By acquiring a representative background image of a sample for which the analytes comprised therein are to be spatially determined in the further course, as well as by simulating signals of the markers using the representative background image, as well as a known point spread function of the microscope, an annotated data set can be created in a simple manner and with sufficient accuracy, so that a suitable annotated data set corresponding to the sample with which a suitable processing model can be trained is made available.

Due to the fact that generative models are particularly well suited for artificially creating images, the generation of an annotated data set with a generative model in a particularly efficient manner results in a high-quality annotated data set.

By capturing reference images with a background image as well as at least one image for each background in which each analyte to be identified is marked, an annotated data set can be created for a respective background image accordingly, because all analytes to be identified are marked via the images and can thus be easily distinguished from the background image.

By carrying out a classical method for the spatial recognition of analytes prior to the creation of the annotated data set, a particularly realistic annotated data set can be created. The creation of the annotated data set in this case is in fact very computationally intensive because the classical evaluation methods are very computationally intensive; since in this case, each of the target series determined by means of the classical method contains images from a result feature space, a matching is nevertheless particularly reliable here.

By subtracting the image signals of a representative background image from the image signals of the image series, the processing model can disregard the different backgrounds in the different image areas and only needs to be trained according to the signal series that occur. Therefore, the processing model should be able to be trained faster by first subtracting the representative background image.

Preferably, the training of the processing model is a completely new learning of the processing model or a transfer learning of a pre-trained processing model. The pre-trained processing model can, for example, be selected from a set of pre-trained processing models on the basis of contextual information.

By making the processing model a pre-trained processing model, the total time spent on training can be significantly reduced. At the same time, this leads to highly specific processing models with a high accuracy in the assignment of signal components.

Another aspect of the invention relates to a method for determining a signal composition of signal series of an image series. The image series is generated by marking analytes with markers in a plurality of coloring rounds and detecting the markers with a camera. The camera captures an image of the image series in each coloring round, the markers are selected in such a way that the signal series of analytes in an image area comprise colored and uncolored signals across the image series, and the signal series of the different analyte types each have a specific sequence of colored signals and uncolored signals, and the different analyte types can be identified on the basis of the specific sequence. The method comprises the following steps: receiving the signal series; importing a codebook, wherein the codebook comprises a target series for all signal components, the target series comprises analyte target series, and the analyte target series has a sequence of true and false values according to the specific sequences of the signal series of the different analyte types; and determining the signal composition for each of the signal series, wherein a signal portion of the respective signal series is assigned to the target series of the codebook in accordance with the signal composition.

According to the present invention, a codebook for each analyte type comprises a sequence of markers that couple to the respective analyte type in the respective coloring rounds.

In conventional methods for the identification of analytes in an image series, bright pixels are first identified across the image series, a signal series is created from the sequence of bright pixels, and the signal series is directly matched with signal series in a codebook. As a result, the analysis includes the analyte type that best matches the particular signal series. No methods are known from prior art that match signal series with a mixture of a plurality of analyte types and that output, for example, a mixing ratio of a plurality of analyte types.

The inventors have realized that, in the event of multiple signal series, contributions from a plurality of analytes can be seen. This means that the analytes are so close together in the sample that they are mapped onto the same image area due to the resolving power of a microscope. Due to the fact that the method for determining a signal composition based on the target series of a codebook assigns a signal portion to different target series, respectively, the present method makes it possible to analyze signal series of image areas and to identify analyte types even if they are mapped onto the same image area. This is not possible with the prior art cited above.

Preferably, the signal composition is determined with a signal portion function. The signal portion function detects a difference between the respective signal series and a linear combination of a plurality of the target series. The signal composition is determined by optimizing the signal portion function based on the signal portions.

By determining the signal composition by means of a signal portion function to be optimized, the signal composition can be determined in a simple manner.

Preferably, the optimization of the signal portion function is performed by means of one of the following algorithms: a non-negative matrix factorization, a main component analysis, a discriminant function, a singular value decomposition, or a classical optimization method, in particular a convex optimization, a non-convex optimization, a concave optimization, a linear optimization, or a non-linear optimization, with the classical optimization method being carried out with or without constraints, preferably with constraints, in particular boundary conditions.

By suitably optimizing the signal portion function, a signal composition can be correctly determined with the algorithms mentioned.

Preferably, the optimization is performed with predetermined boundary conditions. The boundary conditions include, for example: the signal portions can be non-negative, the entries in the target series can be non-negative, a number of the colored signals in a target series is specified for all analyte types in the codebook, for example as a fixed value or as an interval, or the number of the colored signals is specified individually for each of the target series.

During an experiment, the number of different analyte types in the codebook exceeds the number of measured values, i.e., the image signals across the signal series, either per pixel or per image area. Therefore, the mathematical optimization problem does not have a unique solution, which is also spoken of as the problem being stated poorly. By choosing suitable boundary conditions as mentioned above, the unsolvable problem can be transformed into a solvable problem. In this case, for example, the boundary conditions described are predetermined by the physical boundary conditions; it makes no physical sense, for example, to assign a negative value to the signal portions; it also makes no sense for the entries of the target series to have no negative entries.

Preferably, the optimization is performed by using a regularization. The regularization parameters of the regularization include, for example: a predetermined maximum number of different signal components, an expected number of analyte types, a limitation of the ability of the analyte types of the codebook to combine with one another, and a limitation of the optimization to sparse solutions, i.e., only a few of the different target series of the codebook have signal portions at any given time.

By initiating a regularization, the mathematically unsolvable or poorly solvable problem can be modified in such a way that it becomes mathematically solvable.

Preferably, the determination of a signal composition further comprises the following steps: entering the signal series into a processing model, wherein the processing model has been trained, for example, according to any of the methods described above for training a machine learning system with a processing model, to provide a result output from which the signal portion of the respective signal series is determined for each signal component.

By determining the signal composition by means of a processing model, such as a neural network, the signal composition can be determined quickly and efficiently.

Preferably, the processing model is a classification model and the result output for each signal series is a probability distribution across the signal components of the codebook, each indicating a probability of belonging to one of the signal components and determining the signal portion on the basis of the probability distribution.

Since the processing model is a classification model that outputs a probability distribution across the signal components of the codebook, it is possible, for example, to identify all signal components with a probability above a threshold value as being included in the signal series and to determine a signal portion based in each case on the level of probability. By using the classification model, this assignment is particularly simple. In addition, the result can also be used to directly determine how certain the processing model is about the assignment of the signal component, which allows the user, if necessary, to check the corresponding assignment in case of a doubtful assignment, which is particularly desirable.

Preferably, the result output is based on a multiplication of a layer output of the processing model by an analyte matrix. The analyte matrix is based on the target series of the codebook. The result output provides a value for each of the signal components from which the signal portion is determined.

By implementing the result output by means of a simple matrix multiplication, the result output can be determined in a particularly simple way. Since the multiplication with the analyte matrix is implemented in the network, the result output can be calculated particularly efficiently, for example on a graphics card or a special acceleration card for machine learning, for example a tensor processor or an application-specific chip. Furthermore, the result output is implemented only by means of a matrix multiplication in the last convolutional layer. It is very easy to switch the computation of the result output to a new codebook by replacing the analyte matrix with a different analyte matrix and without having to retrain the processing model. If the processing model has been nonspecifically trained to recognize colored and uncolored signals, an analyte-agnostic model has thus been trained that can be easily switched to new analyte matrices and thus to new specific sequences or new analyte-specific samples.

Preferably, the processing model is a classification model, wherein the layer output comprises a probability distribution that assigns to each image signal of a signal series a probability of being a colored signal. The target series are bit series that comprise a true value for each expected colored signal and that comprise a false value for each expected uncolored signal. For each signal series, the result output comprises a sum of the probability values of the layer output corresponding to a true value of the target series. The signal portion is determined on the basis of the sum.

Since the processing model is a classification model that outputs the probability that the respective image signal is a colored signal for each of the image signals of a signal series, and since the probabilities are multiplied with the analyte matrix in the matrix multiplication in such a way that, for each signal component, only the probabilities corresponding to the true values of the respective signal components are added up, a higher value, i.e. a higher output sum of the probability values, means that more and higher probabilities correspond respectively to the true values of the target series. Thus, a higher sum indicates that it is highly probable that many of the true values of the target series in the signal series are colored signals. The sum thus provides a simple indication about which of the signal components have portions in the signals of the signal series.

Preferably, the processing model is an embedding model. The embedding model determines the embeddings of the signal series and target series in an embedding space such that the layer output is a result embedding, and the analyte matrix is based on the embeddings of the target series. The embedding model was trained to map signal series of a certain analyte type and their corresponding target series to the embedding space such that the different embeddings corresponding to the same signal component have the smallest possible distance in the embedding space and such that the embeddings corresponding to different signal components have the largest possible distance. Furthermore, the embeddings of signal series with signal portions from a plurality of signal components should have as small a distance as possible to the embeddings of the respective plurality of signal components and as large a distance as possible to the embeddings of the remaining signal components.

Training the embedding model in such a way that signal series with signal portions from a plurality of signal components are also embedded so that a distance to the embeddings in the respective plurality of signal components is minimal achieves the fact that this proximity also exists in the embedding space for signal series with a plurality of signal components, which have a particular proximity to the signal series of the respective signal components in the feature space, i.e., short distances in the feature space are mapped onto short distances in the embedding space, which makes it particularly easy to determine the respective signal components, which make up the signal series with the plurality of signal components, on the basis of the determined distances in the embedding space.

Preferably, during the training of the processing model, an annotated data set was used that comprises training signal series and the corresponding target series for a plurality of signal components to be identified, which each correspond, for example, to one analyte type. During the training, training signal series from different signal components, for example different analyte types, are linearly combined and the linear combination is input into the processing model. The corresponding target series are linearly combined accordingly and used in the training for calculating the objective function.

By generating training signal series that are comprised of signal series from a plurality of signal components, the processing model can be specifically trained to recognize such mixed signal series. On the one hand, this is advantageous for a processing model that is implemented as an indication model, because this makes it possible to specifically achieve a suitable implementation in the processing model. On the other hand, this also makes it possible to specifically achieve the embedding of the mixed signal series as described above.

Preferably, the determination of a signal composition further comprises the following steps: clustering the extracted signal series with a cluster analysis algorithm, wherein a number of predetermined clusters is at least equal to a number of the signal components; determining a cluster center for each of the clusters; determining at least one target cluster center for each of the signal components based on the target series; determining the cluster distances between the cluster center and the target cluster centers for each of the cluster centers; assigning, based on said cluster distances, the clusters to one of the signal components; determining the distance to the respective cluster centers for each of the signal series; and determining the signal portion based on the distances.

A cluster analysis makes it possible to determine clusters for each of the signal components. For signal series that are comprised of a plurality of signal components, a distance to the signal components that the signal series is comprised of should be minimized in the cluster analysis space.

Thus, by determining the respective minimum distances to the respective cluster centers, a portion of the signal components in the respective signal series can be determined.

Preferably, the respective distance is a Euclidean distance in the cluster analysis space; alternatively, the distance may, for example, also depend on a dispersion of the values within a cluster, e.g. may be normalized based on the dispersion. Furthermore, when determining the distance, an entropy of the respective signal series or an entropy of a distance vector may also be taken into account, wherein the distance vector is just the vector between the position vector of the signal series in the cluster analysis space and the position vector of the cluster center in the cluster analysis space.

Preferably, n of the coloring rounds correspond to one marking round each and each analyte is detected in only one of the n coloring rounds of a marking round, i.e., one analyte is coupled to only one of the n markers, wherein the n markers are embodied in such a way that only one of the n markers is coupled to each of the analyte types in each marking round and each of the n markers is recorded in a different color contrast. When determining the signal composition, for example, it is taken into account as a boundary condition that an analyte is marked with a marker in only one of the n coloring rounds of a marking round.

By entering as a boundary condition that each of the analyte types is coupled with a marker in only one of the n coloring rounds of a marking round and thus can be a colored signal in only one of the n coloring rounds of a marking round, it can be directly concluded during the optimization that a plurality of analyte types generate image signals in the respective signal series, for example, if more than one colored signal is obtained in an image area or in a signal series within the n coloring rounds of a marking round.

Preferably, a total of n×m=k coloring rounds are performed and n×m=k images are acquired. A signal series thus comprises k image signals, with each analyte type having a colored signal in a maximum of n of the coloring rounds. When determining the signal composition, for example, it is taken into account as a boundary condition that a maximum of n of the coloring rounds represent a colored signal for each analyte or each signal component, respectively.

By using a maximum number of colored signals as a further boundary condition, the signal composition can be determined in an even more reliable manner.

Preferably, signal component context information is included in the determination of a signal composition. The signal component context information in this case comprises at least one of the following: information about a location of an analyte type in a sample, information about a number of expected analyte types, information about co-localizations of certain analyte types in certain areas of a sample, information about a maximum number of analyte types in certain areas of the sample, a user ID, information about the experiment such as the experiment type and sample type, and information about a background portion in different areas of the sample.

Due to the fact that context information about an identified analyte type or signal component is used in particular when determining the image region, corrections to the determination can be made or errors in the determination can be corrected even after the analyte type of a signal series has been identified.

Preferably, before the determination of a signal composition, in particular before the signal series is input into a processing model, the method further comprises a step of performing a background correction of the image signals of the image series, wherein performing the background correction comprises one or more of the following: a rolling-ball method, a filtering such as a top-hat method, a homomorphic filtering, a low-pass filtering in which the result of the low-pass filtering is subtracted from the signal, or a temporal filtering, a background correction by means of an image-to-image model, a background correction by means of mixed models, a background correction by means of a mean-shift method, a background correction by means of a main component analysis, a background correction by means of a non-negative matrix factorization, or a background correction by means of an excitation of the auto-fluorescence by means of a non-specific laser for all image areas from the image series.

Because the method comprises a background correction, the image signals of the signal series can be separated from the background independently and thus in a better manner, or a computational effort is reduced, for example during the matching, because the background contributions no longer have to be taken into account.

When determining a signal composition for each of the signal series, a background signal component is also preferably considered as another one of the signal components with another signal portion.

By considering a background signal component as well when determining the signal composition, a background can be taken into account particularly well, for example in the linear combination of the signal components including the background signal component, which further improves the identification of the signal components.

Preferably, the background signal component is determined from image signals from image areas surrounding the image area of the signal series, and a portion of the background signal component in the signal series is determined on the basis of the background signal component determined in this way.

By determining the background signal components individually for each signal series based on the surrounding image areas, the background component can be determined particularly reliably according to the surrounding background, which further improves the determination of the signal portions.

When determining a signal composition for each of the signal series, a noise component is also preferably considered as another one of the signal components with another signal portion.

Due to the fact that a noise component is also taken into account when determining the signal composition, a noise from the configuration can be taken into account particularly well, for example in the linear combination of the signal components including the noise component, which further improves a determination of the signal components.

Preferably, the method further comprises a normalizing of the image signals, wherein the normalizing comprises at least one of the following: normalizing the image signals across an entire image; normalizing the image signals across all images of the image series; normalizing the image signals across a signal series; normalizing the image signals across a signal series such that relative signal portions are determined; and/or normalizing the image signals based on a color contrast of the image signals.

By normalizing the image signals prior to the determination of the signal composition, for example, a better correlation with regard to the relative signal portions of different signal components to each other is achieved in the output.

Preferably, for example, the image areas each comprise only one pixel, an area of contiguous pixels, or a contiguous volume in an image stack. For example, the signal series is input into the processing model as a tensor that has entries for each of the pixels in the image area and comprises each of the color rounds. According to one alternative, the values of adjacent pixels in the image area are combined to form entries in the tensor. For example, an average value of adjacent pixels, a maximum value, a minimum value, or a median is input.

By combining a plurality of pixels into one image area, it is possible to reduce the computing power required during the evaluation of the signal series. On the other hand, a pixel-by-pixel evaluation allows, if necessary, for a separation of signals of closely spaced analytes, which would merge with each other and could no longer be separated from each other if the plurality of pixels were combined into one image area with only one single value.

By choosing the size of an image area on the basis of an expected analyte density, it is possible to optimize the required computing power according to an expected analyte density.

Accordingly, a size of an image area may be selected on the basis of an expected analyte density in the sample. Preferably, the size of an image area can vary across the entire image, depending on the expected analyte density in the image area.

When signal series are input into a model, for example the processing model, according to the present invention, either the signal series of individual image areas can be input into the model, also spoken of as the receptive field of the model then comprising only a single image area, or alternatively the receptive field of the model can also comprise signal series from adjacent image areas. In that case, the model processes the signal series of the respective image area on the basis of, among other things, the image signals or the signal series of the other image areas in the receptive field. This is also spoken of as the spatial context being taken into consideration in the processing of the image signals or signal series of the image area, in this case the image signals or the signal series of the adjacent image areas that are part of the receptive field of the model.

A number of image areas in the receptive field may be selected based on, for example, the point spread function of the microscope in such a way that a diameter of the receptive field is not larger than, only slightly larger than, or, for example, twice as large as a diameter of an area onto which a point in a sample is mapped on the basis of the point spread function. For example, the receptive field is 3×3, 5×5, 7×7, 9×9, 13×13, 17×17 image areas in size, but the receptive field may also be 3×3×3, 5×5×5, 7×7×7, 9×9×9, 13×13×13, or even 17×17×17 image areas in size when image stacks are acquired in the coloring rounds.

Preferably, the method comprises a determination of an image region. In particular, the determination of an image region comprises combining adjacent image areas into an image region if the adjacent image areas have signal series with the same signal components, wherein the combining of adjacent image areas comprises, for example, a non-maximum suppression.

By grouping image areas into image regions and determining image region signal series, a computational effort can be significantly reduced when evaluating the image series.

Preferably, the determination of an image region further comprises verifying the image regions, wherein verifying the image regions comprises at least one of the following: separating the image region into two or more image regions if the image region exceeds a maximum size; separating the image regions into two or more image regions if the image regions are each connected to each other only by a few bridge pixels or a shape of the image region indicates that two image regions overlap here; separating the image region based on signal component context information, wherein the signal component context information comprises, for example: information about a size of an image region depending on the analyte types, information about a location of an image region in a sample, information about co-localizations of certain analyte types in certain areas or in a location in a sample, the expected analyte densities depending on a location of the image region in a sample; and the discarding of image regions if an image region falls below a minimum size or has a shape that cannot be reliably assigned to an analyte.

Preferably, the maximum size of the image region is selected depending on the point spread function of an imaging device.

In addition, the maximum size can also be selected on the basis of an expected analyte density in such a way that the maximum size is as small as possible in the case of a high expected analyte density, while larger maximum sizes are permissible in the case of a low expected analyte density. The maximum size can be chosen according to a semantic segmentation of the image.

By choosing the maximum size based on the point spread function of a capturing device, the size of an image region can be optimally matched to an expected expansion of a signal from an analyte. Thus, computational resources are not unnecessarily wasted by analyzing too many signal series, and furthermore, an overly coarse rasterization is prevented as well by choosing the maximum size based on the point spread function.

By separating or disregarding image regions on the basis of certain criteria, the computing power that is required can be considerably reduced when checking whether the signal series of the respective image region is a candidate signal series and when identifying an analyte type of the signal series; in addition, the separation makes it possible to avoid capturing a plurality of analyte types, in particular a plurality of different analyte types in an image region if an expected analyte density is very high.

Preferably, the determination an image region further comprises determining an image region signal series based on the signal series of the image areas that the image region is comprised of, wherein the determination of the signal composition is carried out based on the image region signal series and comprises a combining of image signals from adjacent image areas into a combined image signal of the image region.

Preferably, the determination of an image region is carried out after a signal composition has been determined for each of the signal series.

The fact that the determination of the image regions takes place after the determination of a signal composition ensures that, for example, the image regions can still be separated after the signal composition has been determined, if, for example, so many colored signals are found in an image region that it is possible that image signals from a plurality of analytes were captured in the image region. Accordingly, the separation of the image regions allows for an improved determination of the signal composition of the signal series.

Preferably, the determination of the signal composition comprises a non-maximum suppression.

By using the non-maximum suppression to filter out duplicate determined signal compositions, it is possible to prevent, for example, overlapping or adjacent image areas from being counted twice as analytes that were found.

Preferably, the signal portion indicates a relative portion of the image signal of the respective signal component in the image signals of the signal series.

By outputting the signal portions of the respective signal components as a relative portion of the image signal, it is possible to determine a portion of the respective analytes corresponding to the respective signal components.

Preferably, the signal portion is an absolute portion of the respective signal components in the image signal.

Preferably, the signal composition is first determined with a processing model as described above, the determined signal portions are then used as output values for the above-described optimizing a signal portion function in the form of signal portions of the linear combination, and the signal composition is determined again based on the above-described method for optimizing a signal portion function.

First determining signal components that have a signal portion in the signal series by means of a processing model and then determining the signal portions again by optimizing the signal portion function by means of the optimization method yields a signal portion that is determined much more precisely than if only the processing model were used to determine the signal portions; in addition, the optimization is accelerated considerably, because it is carried out on the basis of the signal portions determined by means of the processing model and the signal components determined by means of the processing model and their signal portions are used as constraints in the optimization, which makes the solution simpler, more solvable, or more uniquely solvable.

Preferably, the method further comprises the following steps: generating an extended annotated data set based on the determined signal portions and performing the method described above for training a machine learning system using at least the extended annotated data set as the annotated data set.

By expanding the annotated data set with verified data, the training of the processing model can be continuously improved.

Preferably, the extraction of the signal series comprises at least one of the following: extracting all image areas from the image series; extracting a random selection of the image areas from the image series; extracting a selection of the image areas from the image series weighted by a structural property of the image areas, for example, with a higher probability for cells, cell nuclei, and bright pixels; extracting image areas exclusively from image areas with a minimum level of image sharpness; and skipping image areas where no analytes are expected.

By deftly extracting the image areas as described above, the effort associated with the evaluation of the image signals of the image series can be reduced considerably.

Preferably, the processing model is selected manually or automatically. The automatic selection is based, for example, on context information, the context information comprising, for example, a sample type, an experiment type, or a user ID.

Preferably, the extraction further comprises the following steps: filtering out candidate signal series from the extracted signal series, wherein a ratio of at least one of the colored and/or uncolored signals of a candidate signal series to at least one other of the colored and/or uncolored signals of the respective signal series is a characteristic ratio and/or a candidate signal series is a characteristic signature comprising the at least one characteristic ratio, such that if the signal series comprises at least one characteristic ratio and/or the characteristic signature, the signal series is considered a candidate signal series.

According to the prior art, pixels are identified in an image series that have an image signal above a certain threshold value. The threshold value is determined locally within each image of the image series. The inventors have realized that, apart from the analytes in an image series which provide particularly bright image signals, there are other analytes whose image signal differs only insignificantly from image signals in an immediate vicinity of the pixels. Such candidate signal series can be identified on the basis of the particular ratio of colored and/or uncolored signals to each other or on the basis of a characteristic signature within a signal series comprising at least one particular ratio. Since the candidate extraction model has been trained to recognize candidate signal series as well as the colored and uncolored signals within a signal series on the basis of the particular ratio or to identify them on the basis of a characteristic signature with the at least one particular ratio, it is also possible by means of the present method to find analytes within a sample which, despite being marked with markers, differ only slightly in at least some of the coloring rounds from a brightness of the other signals of the signal series and a brightness of the surrounding pixels.

Preferably, the candidate signal series filtering is performed with a candidate extraction model, with the candidate extraction model being selected from a set of candidate extraction models based on, for example, a sample type, an experiment type, or a user ID.

By using a machine-learnable candidate extraction model to identify candidate signal series or to identify analyte areas, it is possible to identify analyte areas or candidate signal series in the image series in a particularly efficient manner.

Preferably, the candidate extraction model has been trained to identify the colored and uncolored signals based on at least one particular ratio of one of the colored and/or uncolored signals of the respective signal series to at least one other of the colored and/or uncolored signals of the respective signal series and/or to identify the candidate signal series, respectively, based on a characteristic signature with the at least one particular ratio.

The inventors have realized that the signal series of image areas in which the image signals of analytes are captured each have at least one particular ratio between colored and/or uncolored signals of the respective signal series, which means that a characteristic signature with the at least one particular ratio of the colored and/or uncolored signals is obtained for the candidate signal series. Based on the particular ratio, colored and uncolored signals in a signal series can be identified and thus a number of colored signals in a signal series can be determined. Based on the particular ratio or based on the characteristic signature, a candidate extraction model can be trained to identify the colored and uncolored signals as well as the candidate signal series in signal series of an image series, i.e., the candidate extraction model learns to recognize certain patterns in the image signals of the signal series.

By first filtering out the signal series of a candidate area from all signal series before matching the respective signal series with corresponding target (bit) series to determine the signal composition of the respective candidate area or candidate signal series, the computational effort used to determine an analyte type of a candidate area can be significantly reduced since considerably fewer signal series have to be matched with a codebook.

Preferably, the candidate extraction model is a semantic segmentation model that outputs a semantic segmentation mask that assigns to each image area a semantic class that indicates whether or not the image area captures image signals of an analyte.

Preferably, the segmentation mask comprises more than two classes. For example, a class in which candidate signal series are not searched for in the first place, a class that assigns the image areas to the background, and a class with image areas in which candidate signal series were found. Alternatively, the segmentation mask may also comprise a plurality of classes in which candidate signal series can be found, each of the plurality of classes comprising, for example, only particular candidate signal series or a particular ratio of different analyte types to each other.

Since the candidate extraction model is a semantic segmentation model, a class of the respective image area assigned according to the semantic segmentation model can be used in a determination of the signal composition following the identification of the candidate signal series according to the class assigned by the semantic segmentation model to match the signal series to the codebook only on the basis of the class or to compare it with the target bit series of the codebook, which can save further computing resources during the matching process, since, for example, fewer target bit series have to be matched.

The fact that the segmentation mask comprises more than two classes means, for example, that image areas outside cells can be recognized directly by the model, in which case no search for candidate signal series is carried out in these image areas at all, thus further accelerating the process and saving computing power.

Preferably, the candidate extraction model is a patch classifier that uses a sliding window method to assign the value to each image area.

Preferably, the candidate extraction model is a fully convolutional network and has been trained as a classification model with fully connected layers with signal series of individual image areas, wherein after the training, the classification model is transformed, through replacement of the fully connected layers with convolutional layers, into the fully convolutional network that processes the signal series of all image areas from the image series simultaneously.

By using a classification model with fully connected layers to train the candidate extraction model, the required computing capacity is significantly reduced during the training, so that the training can be accelerated considerably and so that the optimized model parameters of the classification model can then be used in the fully convolutional network. Due to the fact that a predominant portion of the image areas from the image series do not capture signals from analytes and thus belong to the background image areas, training as a fully convolutional network, into which complete images would always be imported, would result in a very unbalanced training, since a ratio between signal series from background image areas and signal series with image signals from analytes would be dominated by the signal series from background image areas. Therefore, the training as a fully connected network allows the training data to be balanced by an appropriate balanced selection of signal series from background image areas and image areas capturing the signals from analytes, so that the identification of candidate signal series is also sufficiently trained. A fully convolutional network can then be used in the inference, which in turn increases a throughput of the network.

According to one alternative, the candidate extraction model can also be trained directly as a fully convolutional network.

Preferably, the candidate extraction model is an image-to-image model that performs an image-to-image mapping that assigns to each image area a distance value indicating how far the image area is from the closest image area having a candidate signal series, or that assigns to each pixel a probability of being an image area having a candidate signal series.

Due to the fact that the candidate extraction model is an image-to-image model, a threshold can be easily set in the identification of the signal series to be used for matching the signal series with the target series of a codebook based on the target output, so that, for example, signal series with the smallest possible distance value or the highest possible probability value are first selected in the inference of the model and successively inferred with an increasing distance value or a decreasing probability value until a number of analytes found corresponds to an expected number of analytes found.

Preferably, the candidate extraction model is implemented as a detection model and outputs a list of image areas that capture the image signals of an analyte.

The image coordinates include spatial and temporal components since the image series has both spatial and temporal coordinates.

Due to the fact that the candidate extraction model is implemented as a detection model, the output of the candidate extraction model comprises very little data, especially in the case of low numbers, and therefore little data is consumed.

Preferably, before checking whether the signal series is a candidate signal series, the method further comprises a step of “transforming the signal series by means of a main axis transformation or a singular value decomposition,” wherein the transformed signal series is used to check whether the signal series is a candidate signal series.

By inputting transformed signal series into the candidate extraction model, for example, certain background components that can be easily eliminated from the transformed signal series by using the main axis transformation or singular value decomposition can already be virtually eliminated by the transformation before they are input into the model, making it easier for the model to detect colored and uncolored signals or candidate signal series.

Preferably, the image areas are either each only one pixel, an area of contiguous pixels, or a contiguous volume in an image stack, wherein the image signals of the image areas are input into the candidate extraction model, for example, as a tensor.

By combining a plurality of pixels into one image area, it is possible to reduce the computing power required during the evaluation of the signal series. On the other hand, a pixel-by-pixel evaluation makes it possible, if necessary, to separate image areas that are close to each other and would merge with each other if the plurality of pixels were combined.

Accordingly, a size of an image area may be selected on the basis of an expected analyte density in the sample. Preferably, the size of an image area can vary across the entire image, depending on the expected analyte density in the image area.

By choosing the size of an image area on the basis of an expected analyte density, it is possible to optimize the required computing power according to an expected analyte density.

Preferably, the processing model and the candidate extraction model form a common assignment model with a common input layer.

Preferably, a plurality of the candidate extraction model and processing model layers, comprising the common input layer, form a common input master in which the signal series for the candidate extraction model and the processing model are processed together.

Preferably, the signal series are first processed by the candidate extraction model and the signal series identified as candidate signal series are then processed by the processing model to determine the signal composition of the candidate signal series. Alternatively, the signal series are processed independently of each other in the two models.

Because the extraction of the candidate signal series and the assignment of the signal composition of the candidate signal series are implemented in a common model with a common input layer, a processing of the signal series can be simplified by the fact/to the effect that only one model, the assignment model, needs to be served.

By having the processing model and the candidate extraction model share the common input master, the computations performed in the common input master only need to be carried out once, which provides speed advantages.

Preferably, the outputs of the two models of the assignment model are combined in a final assignment step independently of the assignment model.

Alternatively, the output of the two models is combined in an output layer of the assignment model such that signal series not identified as candidate signal series by the candidate extraction model are automatically assigned to a background to which they correspond, and the identified candidate signal series become the signal composition according to the assignment of the processing model.

By combining the outputs of the two models of the assignment model in a final output layer, a possibly time-consuming assignment outside the assignment model can be omitted, which further speeds up the assignment.

### BRIEF SUMMARY OF THE FIGURES

The invention is explained in more detail below with reference to the examples shown in the drawings. The drawings show the following:

FIG. 1 schematically illustrates a system for use with the method for identifying analytes in an image series according to one embodiment;

FIG. 2 schematically illustrates a device for use with the method according to one embodiment;

FIG. 3 schematically illustrates a method for generating an image series by marking analytes with markers in a plurality of coloring rounds and detecting the markers with a camera, as is performed before identifying analytes in the image series;

FIG. 4 schematically illustrates a method for assigning a result class;

FIG. 5 schematically illustrates a method for assigning a result class;

FIG. 6 schematically illustrates a method for assigning a result class;

FIG. 7 schematically illustrates a processing model as may be used according to several of the embodiments;

FIG. 8 schematically illustrates measurement data of the kind that is analyzed in the various methods of the various embodiments;

FIG. 9 schematically illustrates processes of a method according to a further embodiment;

FIG. 10 schematically illustrates processes of a method according to a further embodiment;

### DETAILED DESCRIPTION OF THE EMBODIMENTS

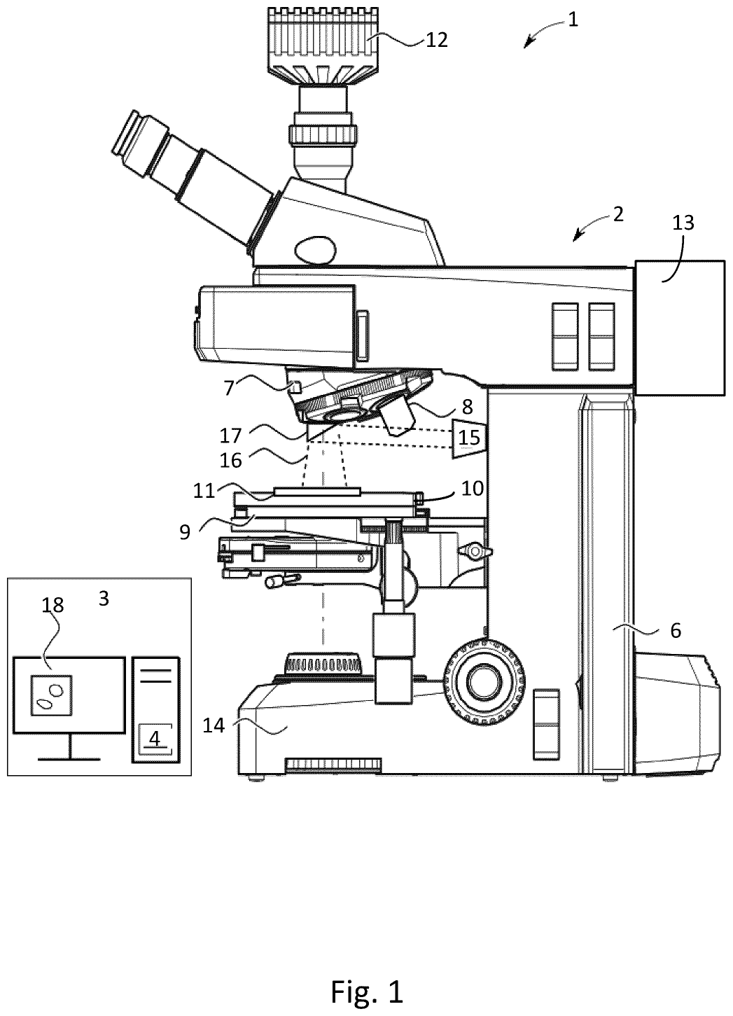

One embodiment of an analyte data evaluation system 1 comprises a microscope 2, a control device 3, and an evaluation device 4. The microscope 2 is communicatively coupled to the evaluation device 4 (for example, with a wired or wireless communication link). The evaluation device 4 can evaluate microscope images 5 captured with the microscope 2 (FIG. 1). If the analyte data evaluation system comprises a processing model, it is also referred to as a machine learning system.

The microscope 2 is a light microscope. The microscope 2 comprises a stand 6 which includes further microscope components. The further microscope components are, in particular, an objective changer or nosepiece 7 with a mounted objective 8, a specimen stage 9 with a holding frame 10 for holding a specimen carrier 11, and a microscope camera 12.

If a specimen is clamped into the specimen carrier 11 and the objective 8 is pivoted into the microscope beam path, a fluorescent illumination device 13 can illuminate the specimen for fluorescence imaging purposes, and the microscope camera 12 receives the fluorescent light as a detection light from the clamped specimen and can acquire a microscope image 5 in a fluorescence contrast. If the microscope 2 is to be used for a transmitted light microscopy, a transmitted light illumination device 14 can be used to illuminate the sample. The microscope camera 12 receives the detection light after it has passed through the clamped sample and acquires a microscope image 5. Samples can be any objects, fluids, or structures.

Optionally, the microscope 2 comprises an overview camera 15 for acquiring overview images of a sample environment. The overview images show, for example, the specimen carrier 11. A field of view 16 of the overview camera 15 is larger than a field of view 16 when the microscope camera 12 acquires a microscope image 5. The overview camera 15 looks at the specimen carrier 11 by means of a mirror 17. The mirror 17 is arranged on the nosepiece 7 and can be selected instead of the objective 8.

According to this embodiment, the control device 3, as shown schematically in FIG. 1, comprises a screen 18 and the evaluation device 4. The control device 3 is configured to control the microscope 2 for acquiring image series 19 of microscope images 5 and to store the image series 19 acquired by the microscope camera 12 on a memory module 20 of the evaluation device 4 and to display them on the screen 18 as needed. The acquired microscope images 5 are then further processed by the evaluation device 4.

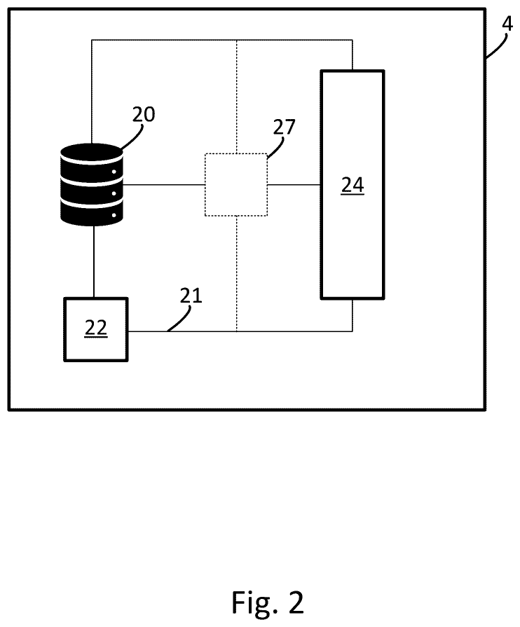

The evaluation device 4, as shown schematically in FIG. 2, comprises various modules which exchange data via channels 21. The channels 21 are logical data connections between the individual modules. The modules can be software modules or hardware modules.

The evaluation device 4 comprises the memory module 20. The memory module 20 stores the images acquired by the microscope 2 and manages the data to be evaluated in the evaluation device 4.

The evaluation device 4 comprises the memory module 20, by means of which image data from the image series 19 is held and stored. A control module 22 reads the image data from the image series 19 as well as a code book 23 from the memory module 20 and sends the image data and the code book 23 to a processing module 24. According to one embodiment, the control module 22 reads the signal series 31 of each image area of the image series 19 and inputs them into the processing module 24.

According to one embodiment, the processing module 24 comprises a processing model, such as a classification model, that is implemented as a neural network. The processing module 24 receives the signal series 31 from the control module 22 and, as a result output, either outputs signal portions of signal components for each of the input signal series 31, or the result output, for each of the signal components, outputs a probability that the respective signal component has a signal portion in the signal series 31.

The control module 22 receives the result output from the processing module 24 and stores it in the memory module 20.

As part of the training of the classification model, the control module 22 reads an annotated data set from the memory module 20 and inputs it into the processing module 24, for example, by using a stochastic gradient descent method. Based on the result outputs of the classification model and target outputs contained in the annotated data set, the control module 22 calculates an objective function and optimizes the objective function by adjusting model parameters of the classification model.

Once the classification model is fully trained, the control module 22 stores the determined model parameters in the memory module 20. In addition to the model parameters, the control module 22 may also store contextual information about the acquired images 5.

In each case, the processing model can be implemented as a neural network, a convolutional neural network (CNN), a multi-layer perceptron (MLP), or a sequential network, for example can be a recurrent neural network (RNN) or be implemented as a transformer network.

If the processing model is implemented as a sequential network, the signal series 31 are not input into the respective model as a whole, but rather the image signals of the signal series 31 are input into the model individually. If the model is a convolutional network and is implemented as a sequential network, then the model first sees the image 5 of a first coloring round, then the image 5 of a second coloring round and then, step by step, the images 5 of the following coloring rounds. In a coloring round N, the model only receives the image from round N and has an internal state that internally codes or stores the images 5 from rounds 1 to N−1. In round N, the model then processes the internal state with the image 5 from the coloring round N.

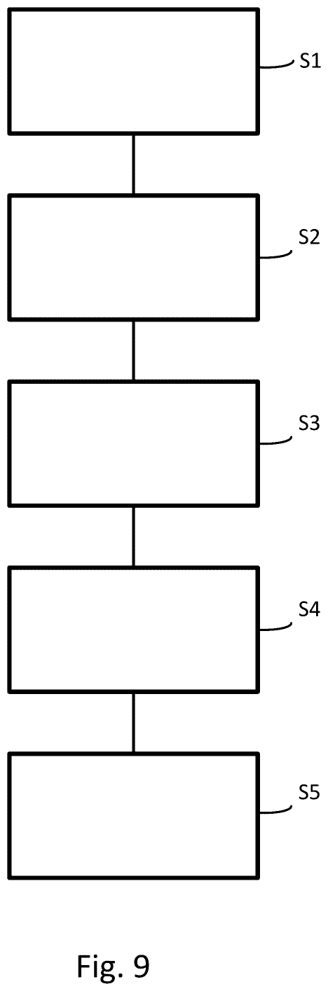

A method of operating the analyte data evaluation system 1 (FIG. 9) is described below.

In the method described for operating the analyte data evaluation system 1, annotated data sets are first generated in a step S1. For this purpose, the microscope camera 12 first acquires an image series 19. To acquire the image series 19, the analytes 39 in a sample are marked in a plurality of coloring rounds such that, for image areas that capture image signals from an analyte 39, a signal series 31 comprising colored signals and uncolored signals is obtained across the image series 19, wherein the markers are selected such that a sequence of colored signals and uncolored signals corresponding to a target bit series 35 of the analyte types in the codebook is obtained for the signal series 31 of a particular analyte type.

In accordance with the present invention, the markers are coupled to analytes 39 and then captured by the microscope camera 12. When coupling the markers to the analytes 39, different analytes 39 may be marked with markers having different fluorescent dyes. For example, if n different fluorescent dyes are used, a number of n images 5 are acquired after the coupling. The n images 5 are each acquired with a different fluorescence contrast corresponding to the number n of different fluorescent dyes. Each of these n images corresponds to one coloring round. After the acquisition of the n images 5, the markers are decoupled from the analytes 39 again. A coupling process as well as the acquisition of the n coloring rounds together with the decoupling of the markers is also called a marking round. After the markers have been decoupled from the analytes 39 again, the analytes 39 can be marked again with new markers in a new marking round. When re-coupling markers to analytes 39, differently colored markers can each couple to analytes 39 this time. Some of the analytes 39 to be identified may not be marked with a marker at all in some of the different individual marking rounds. A signal series 31 expected for a particular analyte 39 or a particular analyte type results from the resulting patterns of colored and uncolored signals or from colored and uncolored signals, in each case in relation to a fluorescence color. These signal series to be expected are summarized in the codebook 23 for all analyte types to be identified, with the markers in the respective marking rounds being selected in such a way that just the signal series 31 expected for the respective analyte type is produced.

According to one alternative, only one image 5 can be acquired per marking round by means of a fluorescence image with a broad fluorescence excitation spectrum that excites the fluorescence of all fluorescent dyes used simultaneously. The acquired image 5 is then converted into the respective n fluorescence contrasts by means of filters, so that n images 5 are once again available for n coloring rounds.

According to this embodiment, the codebook comprises target bit series 35, wherein each expected colored signal is assigned a true value and each expected uncolored signal is assigned a false value.

According to another embodiment, only markers with a single fluorescent dye are used per marking round. For this case, the coloring rounds are exactly equal to the marking rounds.

After the acquisition of the image series 19, images 5 of the image series 19 are registered to each other. The registration can be carried out by means of a classical registration algorithm or by means of a trained registration model.

Even though it is described here by way of example that one image 5 is acquired in each of the coloring rounds, a stack of images 5 can also be acquired in each coloring round, in which case the images 5 of the stack must, on the one hand, be registered to each other, and, in addition, the images from different coloring rounds must be registered to each other.

After registering the images 5 of the image series 19 with respect to each other and storing the registered image series 19, the image series 19 may be analyzed by using a classical algorithm for analyzing image series 19 with analytes 39, as described for example in the prior art documents referenced above.

If an image stack is acquired in each coloring round with the acquisition of the image series 19, a signal series 31 may also be extracted for a contiguous volume of pixels in the image stack instead of individual pixels. A signal series 31 according to the present invention always corresponds to an image area, an image area may comprise a single pixel, an area of adjacent pixels, or a volume of adjacent pixels, wherein the image areas in the different images 5 or image stacks of the image series 19 are registered to each other, i.e., the same coordinates in the images 5 show the same objects in the samples.

According to this embodiment, the codebook 23 is present as a collection of target bit series 35.

After the image series 19 has been analyzed, the analyzed signal series 31 may be stored in the memory module 20 as an annotated data set for training the processing model, and a training phase may follow the generation of the annotated data set(s). The control module 22 may store the annotated data set in the memory module 20.

The memory module 20 stores the respective analyte type along with the signal series 31, for example. According to this embodiment, each of the analyte types may be one of the signal components.

According to one alternative, the annotated data set comprises the signal series 31 and the corresponding target bit series 35.

The processing model is trained in step S2.

According to this embodiment, the processing model is trained to determine a signal composition comprising signal portions of the signal components in the signal series 31. According to this embodiment, the processing model is trained to determine a probability distribution across the signal components, in which each signal component is assigned a probability of having a signal portion in the signal series 31.

As described above, the markers in the marking rounds or coloring rounds are selected in such a way that a specific sequence of colored and uncolored signals results for a particular analyte type across the coloring rounds. The processing model must therefore be trained to recognize the specific sequence of colored and uncolored signals in order to identify the different analyte types.

The inventors have realized that colored and uncolored signals, respectively, in signal series of analytes have a characteristic signature comprising at least a particular ratio to each other. In order to distinguish the colored signals from the uncolored signals, the processing model is trained to recognize at least one particular ratio of colored signal to uncolored signal, colored signal to colored signal, uncolored signal to colored signal, or uncolored signal to uncolored signal, or the specific sequence of the colored and uncolored signals, respectively, in a signal series 31 in order to identify the different analyte types.

The particular ratio may be a certain distance or difference between the image signals, a quotient between the image signals, a certain number of image signals with a higher image signal than the others, wherein the ratio may be learned for a normalized image signal or for non-normalized image signals, respectively. While the prior art mainly considers image signals from very bright pixels, the inventors have realized that signal series 31 of pixels that capture image signals of analytes 39 comprise image signals with the particular ratio described above and that the signal series 31 each have the characteristic signature. Analytically, the characteristic signature is difficult to define, as it may be different for different analyte types, but it turns out that (different) neural networks can identify the characteristic signature or the particular ratio very well when adequately trained. Accordingly, neural networks can also be trained not only to identify the characteristic signature, but also to identify the specific sequence of the different analyte types.

In order to distinguish the different analyte types from each other, an annotated data set for each analyte type to be identified must comprise training signal series of an image area that captures the image signals from the respective analyte 39. The colored and uncolored signals of the training signal series have the particular ratio or characteristic signature and/or the sequence specific to the particular analyte type.

According to an alternative embodiment, in which signal portion is assigned to a background signal component as further signal components, the annotated data set may additionally comprise training signal series of background image areas. The background image areas comprise colored signals only sporadically, which are mostly due to non-removed or incorrectly coupled markers.

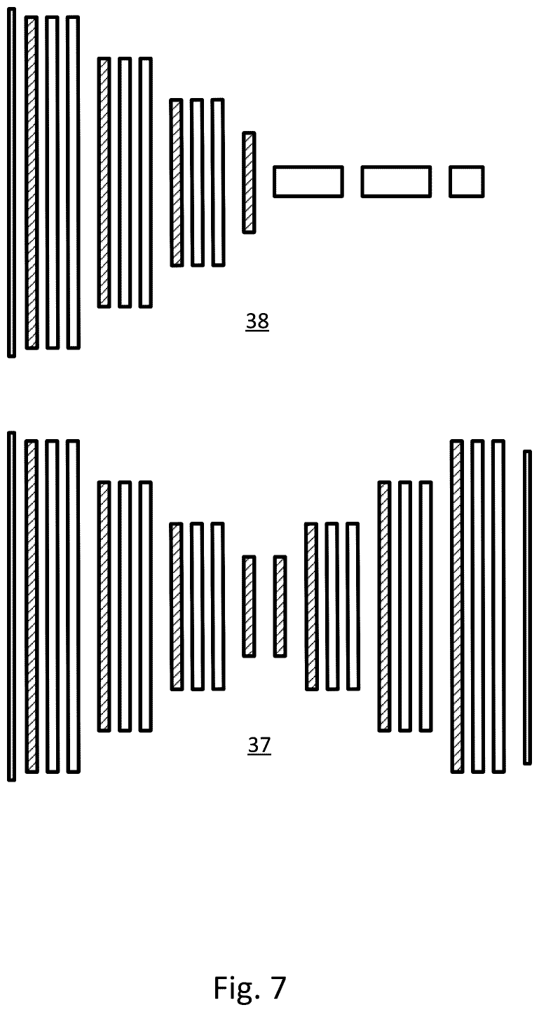

According to the first embodiment, the processing model is a fully convolutional network 37 (see FIG. 7). The processing model is first trained as a classification model, which is a fully connected network 38 with fully connected layers, which is trained with signal series 31 from individual image areas. For this purpose, the control module 22 inputs signal series 31 of the annotated data set into the processing model. The processing model determines a probability distribution for the input signal series 31 that indicates, for each signal component, i.e., for each analyte type and, if applicable, a background signal component, a probability that the input signal series 31 comprises a signal portion of the respective signal component.

According to the present embodiment, the processing model is initially trained only with signal series 31 that can be uniquely assigned to an analyte type or the background.

In turn, the control module 22 controls the training by reading part of the signal series 31 from the annotated data set, transmits the signal series 31 to the classification model, and, using an objective function, detects a difference between the output of the classification model and a target output.

Furthermore, the control module 22 optimizes the objective function based on the model parameters of the classification model.

According to one design of the present embodiment, mixed training signal series can also be constructed from training signal series that are only attributable to a single analyte type by way of augmentation. For this purpose, several of the training signal series, for example 2, are combined with each other by means of a linear combination. The training signal series are then included in the linear combination with their respective signal portion.

Such combined signal series can be comprised of two, three, or more signal series 31, each containing only signal components of one analyte type. Alternatively, a signal component of a background image area with a certain signal portion can be included in the linear combination as well.

If two training signal series from two different analyte types are used, for example, the processing model can be trained to output just these two analyte types as signal components. In this regard, the processing model can either be trained to simply indicate that these two analyte types are signal components of the (combined) signal series. According to one embodiment, however, the processing model can also be trained directly to output the respective signal portion or as described above, a probability distribution 40 across all possible signal components.

In the case where the processing model has been trained to directly output the signal portions of the signal components, the objective function directly detects a difference between the signal portions of the signal components determined by the processing model and the signal portions used in combining the training signal series in the linear combination of the signal components.

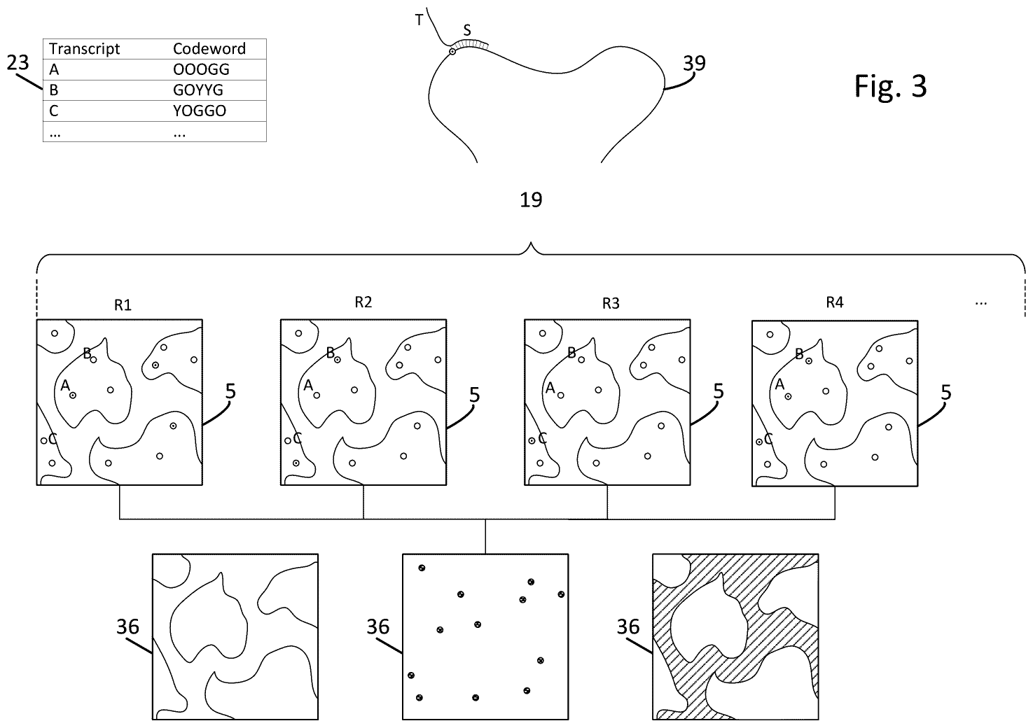

Once the classification model is finished training with the fully connected layers, the fully connected layers are converted to fully convolutional layers. The resulting fully convolutional network 37 can then process a complete image series 31 as input. The output of the trained classification model or the network converted to the fully convolutional network 37 is, for example, the probability distribution 40 described above for each of the image areas from the image series 19 (see FIG. 3, bottom center).

According to another alternative, the annotated data set can also be generated by other means instead of using classical multiomics. The signals of the different markers can, for example, be simulated using a representative background image and a known point spread function of the microscope 2. The codebook 23 is also included in such a simulation.

Alternatively, a generative model can be trained to generate the annotated data set. Since generative models are particularly well suited for generating images 5, a particularly realistic annotated data set can be created with a generative model.

For example, the generative model used may be one of the following models: an active appearance model (AAMs), a generative adversarial network (GANs), a variational autoencoder (VAEs), an auto-regressive model, or a diffusion model.

In addition, one or more reference images, comprising at least one background image, may be acquired as well, and, for each background image, at least one image 5 in which the analytes 39 to be identified are coupled to a marker and the markers are captured in the respective image areas.

Furthermore, if different fluorescent dyes are used in the different coloring rounds, each analyte should be marked with each of the different fluorescent dyes. Of course, any known classical method such as the method described in the aforementioned patent applications EP 2 992 115 B1, WO 2020/254519 A1, and WO 2021/255244 A1 can be used to generate the annotated data set as well.

According to another alternative, the different processing models can be trained to also recognize signal series 31 in which the sequence in which the markers are used in the coloring rounds has been swapped by swapping the sequence of the image signals in the training signal series during the training. It is thus possible to train signal series-agnostic models.

Signal series-agnostic training is particularly useful if no training signal series are yet available for various analyte types to be identified. In this case, the image signals of the signal series 31 would be swapped for training purposes in such a way that a binarization of the image signals of the swapped signal series 31 results in the target bit series 35 that belongs to an analyte type to be identified and for which no training signal series is available.

According to one embodiment, a constructed training signal series may also be constructed for training purposes from a plurality of the training signal series by selecting image signals from a plurality of the training signal series such that a corresponding training signal series with a suitable number of colored and uncolored signals is obtained again. For example, the image signals may be selected in such a way that a binarization results again in a target bit series 35 of the codebook 23.

Alternatively, the sequence of colored and uncolored signals in the constructed training signal series may be arbitrary.

According to the embodiment, after the determination of the objective function, the control module 22 may identify signal series 31 that incorrectly output a probability of a signal component corresponding to an analyte type, even though the input signal series 31 originates from a background image area 25, and from an image area 25 that is within a first predetermined radius around an image area 25 whose signal series 31 actually has a signal component of an analyte type. Since the signal series 31 are randomly selected from the annotated data set, only a few signal series 31 used in training may lie within the first predetermined radius. Correctly classifying such signal series 31 is difficult for the processing model due to the small number in the respective training set. To improve the detection of these misclassified signal series 31, these signal series 31 of background image areas 26 are automatically included in a data set to be trained in a subsequent training round in order to increase their weight in the objective function. This procedure is also called hard-negative mining.

According to a modification, the signal series 31 of pixels that are within a second predetermined radius, which is smaller than the first predetermined radius, immediately adjacent to an image area 25 that correctly captures a candidate signal series, can optionally not be included in the following training round as part of the hard negative mining. According to the point spread function of microscopes 2, marker signals typically span multiple pixels. If signal series 31 of pixels within the second predetermined radius were now also used for hard-negative mining purposes, this would result in a blurring of the class boundaries, which should be avoided.

When training the processing model, a pre-trained model can be selected from a set of pre-trained models and the pre-trained model can be adapted to a new experiment by means of transfer learning.

Alternatively, the signal components can be identified in two steps. First, the signal series 31 is binarized. Then, a matching or comparison to the target bit series 35 of the codebook 23 is carried out. If the assignment of the analyte type takes place in two steps, the processing model must be trained as a binarization model. The binarization model maps the image signals of the candidate signal series, i.e., the colored signals and the uncolored signals, to bit values, i.e., true and false. When the binarization model is trained, the acquired signal series 31 are mapped onto bit series.

A result output of the binarization model is an output bit series; the objective function detects a difference between the target bit series 35 contained in the annotated data set and the output bit series.

Alternatively, the binarization model may be designed to output a probability of being a colored signal for each image signal in the signal series 31.

As also described above with reference to the classification model, a combined signal series can also be generated from a plurality of signal series 31 by means of a linear combination when the binarization model is being trained; when training with combined signal series, the target bit series 35 must also be combined in such a way that all expected colored signals correspond to a true value.

The binarization of the signal series 31 can also be performed by means of a heuristic approach. Alternatively, a generative model can also perform the mapping into the binary space.

For example, the generative model used may be one of the following models: an active appearance model (AAMs), a generative adversarial network (GANs), a variational autoencoder (VAEs), an auto-regressive model, or a diffusion model.

In addition to the analyte types to be identified, the signal components also comprise at least one class that is representative for the signal series 31 of image areas that must be assigned to the background. Such an assignment to the background always takes place if, for example, a match to the target bit series 35 is very poor, or also if the probability output by the processing model for all signal components corresponding to the analyte types to be identified results in a very poor value, i.e., a very low probability.

According to one alternative, the processing model is an embedding model. An embedding model embeds input into an embedding space. In particular, the embedding space must be large enough so that a mapping to be learned by the embedding model from a signal space of the signal series 31 and/or a binary space of the target bit series 35 into the embedding space satisfies the following conditions: An objective function of the embedding model is optimized such that embeddings corresponding to the same result class have the smallest possible distance in the embedding space. This means that a distance between embeddings of the signal series 31 and the corresponding target bit series 35 of the same signal component in the annotated data set is minimized by performing a suitable adjustment of the model parameters of the embedding model, as is a distance between embeddings of two signal series 31 belonging to the same signal component.

At the same time, the objective function is chosen or optimized so that a distance between embeddings belonging to different result classes has the largest possible distance in the embedding space.

According to a further embodiment, the training of the embedding model may further be optimized such that the embeddings of the signal series 31 comprising image signals from a plurality of signal components, in particular a plurality of analyte types, are embedded in the embedding space in such a way that their distance to the embeddings of the signal components with a non-zero signal portion is always smaller than the distance to the embeddings of signal components whose signal portion is very small or zero.

Since the signal series 31 and the target bit series 35 are located in different spaces, it may be difficult to suitably optimize the embeddings of the signal series 31 and the target bit series 35 simultaneously. Therefore, the embedding model preferably has two different input paths or processing paths for the signal series 31 and the target bit series 35, which can further reduce a distance between the embeddings of the signal series 31 and the target bit series 35, further improving both the training and the matching during the inference.

According to one alternative, the signal series 31 and the target bit series 35 share the same input path.

According to a further alternative, a candidate group of the candidate objective functions can be calculated first during the training when the objective function is calculated. A candidate objective function differs from the normal objective functions of the models described above in that one of the colored signals is not considered in the calculation of the candidate objective functions. A candidate group corresponds to an input signal series 31; now, as many candidate objective functions are successively computed in the signal series 31 as the input signal series 31 contains colored signals, leaving out a different one of the colored signals in each of the candidate objective functions. Then, an objective function of choice is selected from the candidate group. The objective function of choice is the candidate objective function of the candidate group that has either a second largest, a third largest, or a fourth largest difference between the result output and the target output.

Since it sometimes happens in the signal series 31 that an image signal of a signal series 31 is not recognized as a colored signal, even though, according to a target bit series 35, a colored signal should be present at the corresponding position or in the corresponding coloring round, a model can be specifically trained, by means of candidate objective function or candidate groups and a selection of an objective function of choice, to know that the acquired signal series 31 have errors.

According to a further alternative, the image signals of the training signal series can be swapped during the training in such a way that the colored and uncolored signals in the swapped signal series again correspond to a sequence of another analyte type. A sequence of the binary codes in the target bit series 35 is adjusted accordingly, so that signal series 31 of analyte types for which no measured signal series 31 are available can then be generated as well. This kind of training can be carried out for all models mentioned above.

Once the various models of the analyte data evaluation system 1 have been trained, the inference can be carried out in step S3, i.e., new data can be incorporated and analyzed with the plurality of models of the analyte data evaluation system 1.

According to the first embodiment, images 5 of the image series 19 are acquired first. For this purpose, different markers are coupled to the analytes 39 present in the sample in accordance with a codebook 23 and then an image 5 of the sample is acquired. According to the first embodiment, markers with, for example, n=3 different colors, for example orange, yellow, and green, are coupled to the analytes 39 in each marking round. After the coupling, three images 5 are acquired in three coloring rounds, i.e., one per coloring round. Each of the images 5 is acquired in a different fluorescence contrast with the fluorescence illumination device 13 being operated at different excitation wavelengths or with different filters, in this case, for example, wavelengths to excite fluorescence in orange, yellow, and green. Accordingly, a colored signal is captured for analytes 39 coupled to orange-colored markers in the first coloring round, which is acquired with the orange-colored fluorescence contrast, for example, while an uncolored signal is captured for analytes 39 coupled to yellow or green markers. According to the embodiment, an image 5 is acquired in orange-colored fluorescence contrast in a first coloring round after the coupling, an image is acquired in green fluorescence contrast in a second coloring round after the coupling, and an image 5 is acquired in yellow fluorescence contrast in a third coloring round after the coupling. The codebook 23 shown in FIG. 3 contains a code word coded in the contrast colors of the coloring rounds instead of a target bit series 35. This means that an analyte A is coupled to an orange-colored marker during the first to the third coupling, and to a green marker during the fourth and fifth coupling. Since an image 5 is acquired in orange contrast in the first coloring round after the coupling, an “O” in the code word corresponds to a bit series “100,” a “Y” corresponds to “010” in the bit series and a “G” corresponds to “001” in the bit series. The corresponding analytes A, B, and C are marked in the coloring rounds R1, R2, R3, and R4 of the images 5 of the image series 19 (see FIG. 3).

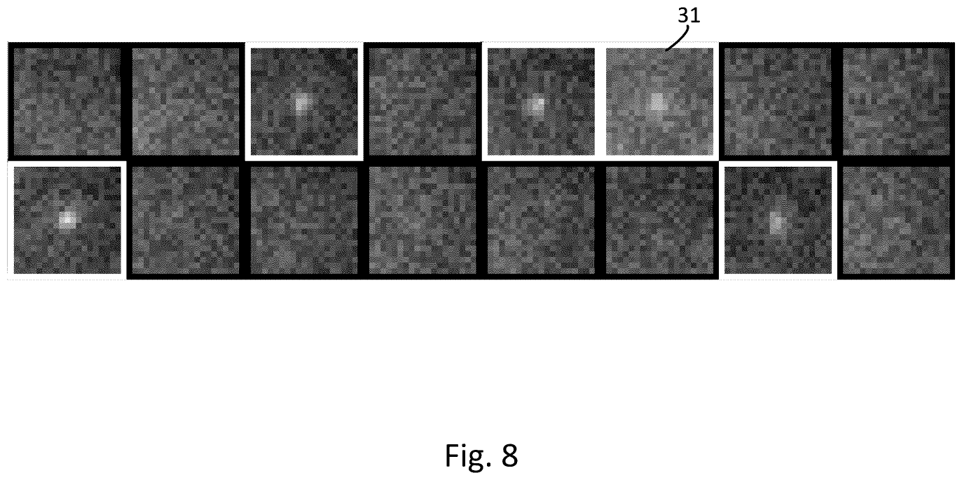

According to one alternative, only a single color contrast, two color contrasts, or more than two color contrasts can be used when capturing the images 5 of the image series 19, with the number of color contrasts preferably corresponding to the number of the different markers used. FIG. 8 schematically shows sections of images 5 of an image series, with the sections outlined in black containing only pixels with uncolored signals, while pixels with colored signals are schematically shown in the center of each of the sections outlined in white. The images 5 in the upper row are acquired with a first color contrast, the images 5 in the lower row are acquired with a second color contrast.

After the image series 19 is acquired, the images 5 of the image series 19 are registered to each other and the image series 19 is stored in the memory module 20.

The control module 22 extracts the signal series 31 and inputs the signal series 31 into the processing model.

The processing module 24 assigns the signal portions of the signal components to the signal series 31. As described above, the assignment of the signal portions of the signal components may also reveal that the signal series 31 does not match any of the analyte types of the codebook 23 and is therefore assigned to the background. If the signal components also include a signal component of a background image area, the processing model assigns this signal component to the signal series 31 accordingly.

As described above, the processing model can directly output the signal portions of the signal components of input signal series 31.

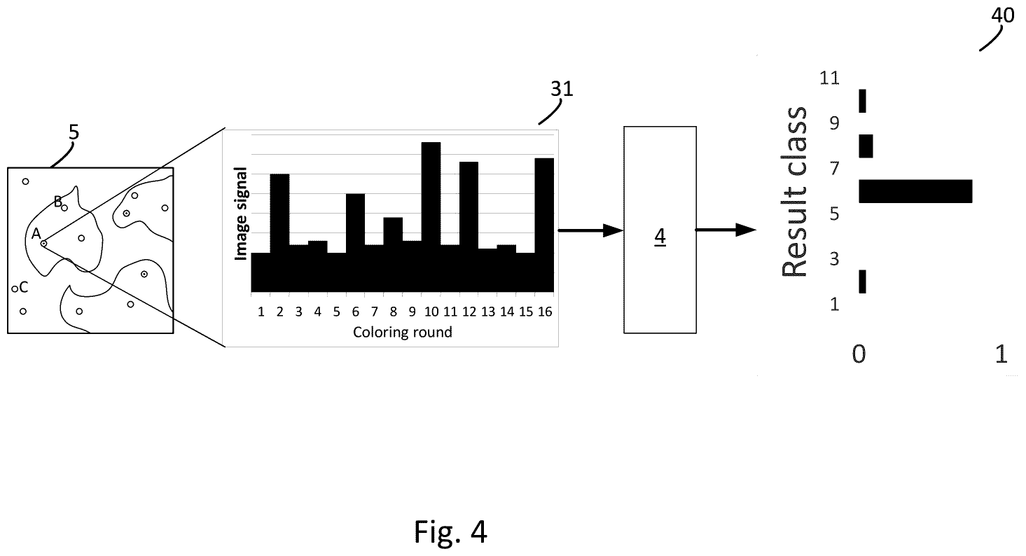

Alternatively, however, the processing model can also output a probability distribution 40 across the signal components (see schematic FIG. 4).

According to a further alternative, on the basis of the probability distribution 40, the processing model outputs—only in binary form and for each of the signal components for which the probability is greater than a threshold value, for example 20, 30, 40, 50 or 60%—that the respective signal component has a signal portion in the signal series 31. In this case, the result output of the processing model is just a vector with a binary entry for each of the signal components.

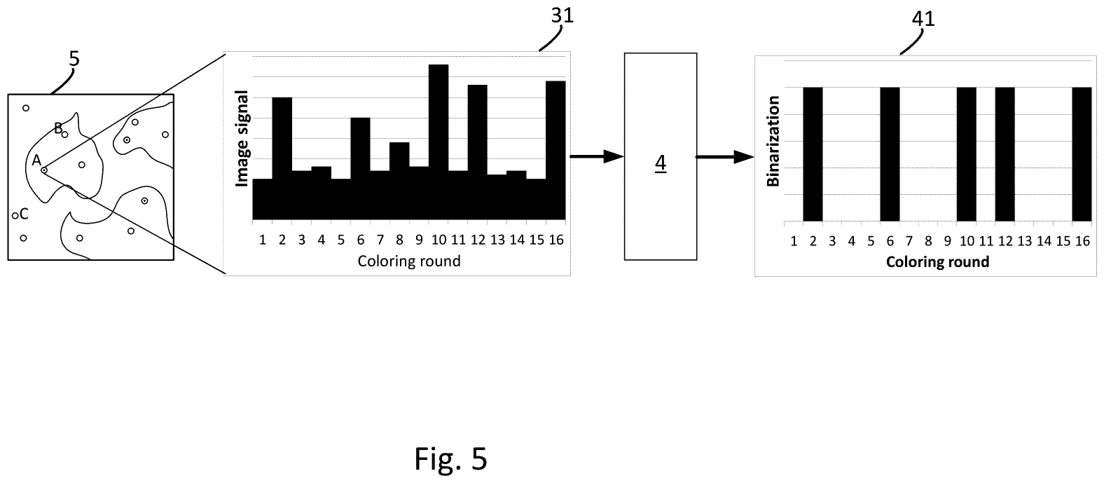

As described above, the processing model may also have been trained to output a binarization 41, also called a bit series, of an input signal series 31 as the result output. The binarization 41 is then used for a comparison or matching with the target bit series 35 of the codebook 23 (see schematic FIG. 5).

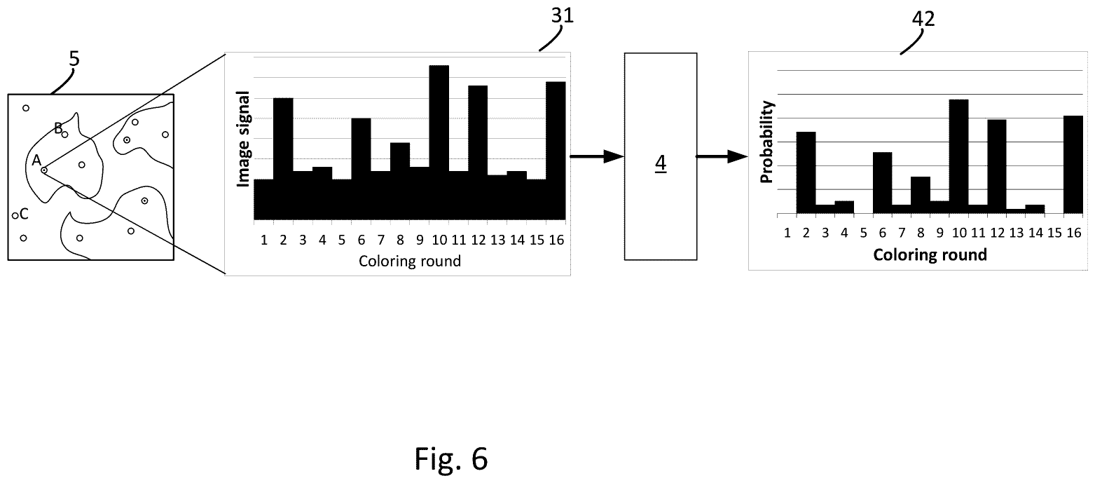

According to a further alternative, the processing model outputs a probability, i.e., a probability series 42, for each image signal in the input signal series 31, with the probability respectively indicating whether the respective image signal is a colored signal (see schematic FIG. 6). The probability series 42 is then used for a matching with the codebook 23.

If the processing model is an embedding model as described above with regard to the training, the matching is carried out in the embedding space. A simple interpretation of the embedding of the signal series 31 is not possible for embedding models. A matching is performed, for example, by determining a distance to the embeddings of the target bit series 35 of the codebook 23 in the embedding space.

According to one alternative, a matching for the alternatives described above in which the processing model outputs either a binarization 41, a probability series 42, or an embedding can be performed by using a matrix multiplication. In the matrix multiplication, the respective result output of the processing model is multiplied by a codebook matrix. The codebook matrix comprises as entries the target bit series 35 of the various analyte types and, if applicable, of the further signal components, for example, of the signal series 31 of the background image areas 26, where all entries of the target bit series 35 typically equal zero. The result of the matrix multiplication is a vector comprising one entry for each signal component. The entry with the highest value then corresponds to a most probable result class.

How to interpret the result of the matrix multiplication will be explained in more detail below on the basis of an example. According to the present example, an experiment comprises 16 coloring rounds.

The different analyte types are coded in such a way that each of the analyte types to be identified in the experiment are marked with a marker in 5 of the 16 coloring rounds. This means that, in the experiment, the image areas that capture image signals from analytes 39 should have exactly 5 colored signals and 11 uncolored signals across the 16 coloring rounds. Correspondingly, the target bit series 35 in the codebook 23 each have five true values and 11 false values.

According to this example, the processing model is the binarization model. The processing model outputs a true value for all colored signals and a false value for all uncolored signals. Thus, the result output is a bit series. The matrix multiplication is now performed in such a way that a scalar product of the result output with the target bit series 35 is calculated for each of the signal components in the codebook matrix. Accordingly, the result of the matrix multiplication for each of the signal components in the codebook matrix is just the scalar product of the respective signal components with the result output. If the scalar product is formed from the result output, which is the binarized signal series, and the corresponding target bit series 35, the result of the scalar product should equal “5,” because in each case a true value in the result output, i.e., a “1,” meets a “1” in the target bit series 35.

Accordingly, for target bit series 35 having a matching true value in only four matching rounds of the 16 coloration rounds, the sum is 4, for target bit series 35 having a matching true value in only three matching rounds of the 16 coloration rounds, the sum is 3, and so on.

According to another example, a combined signal series comprised of signal series 31 from two different analyte types is taken into account. Since the target bit series 35 of different analyte types must differ in at least one bit, for 2 analyte types, a maximum of 4 of the colored signals of the 2 analyte types can occur in the same coloring round.

As above, the experiment has 16 coloring rounds and the analyte types are coded with 5 colored signals. The binarized combined signal series then has 16 entries, of which between 6 and 10 can be a colored signal.

However, depending on how the coding of the analyte types is performed according to the codebook 23, a Hamming distance between the target bit series 35 of the different analyte types may also be more than one bit. According to this example, 2 of the colored signals of the two different analyte types occur in the same coloring round. The remaining 3 colored signals of the two different analyte types occur in different rounds of the 16 coloring rounds. The combined signal series therefore has a total of 8 colored signals across the 16 coloring rounds. Accordingly, the result of the matrix multiplication for more than only the signal components corresponding to the two analyte types on which the combined signal series 31 is based is generally a sum that equals 5, since usually more than just 2 of the target bit series 35 have their 5 colored signals in the coloring rounds of the 8 colored signals of the combined signal series.

In accordance with the two examples described above, more than two of the signal series 31 of the respective analyte types can, of course, be combined to form a combined signal series.

The relative signal portions of the signal components of the combined signal series can also be approximately the same for the different analyte types, i.e., about 50%, but they can also be quite different. It is to be expected, however, that a determination for signal components with signal portions of, for example, 20, 10, or 5% is only possible with great difficulty and inaccuracy.

Preferably, the matrix multiplication with the codebook matrix is implemented in a final layer of the processing model.

In a post-processing step, adjacent image areas can be combined into image regions if, for example, the adjacent image areas each have signal portions of the same signal components.

After the control module 22 has specified the image regions, the specified image regions are checked again. When the image regions are checked, the control module 22 determines, for example, whether the image regions exceed a maximum size, whether the shapes of the specified image regions indicate that two of the image regions should actually be separated from each other, for example because there are only a few bridge pixels between two image regions. In addition, the control module 22 can reject image regions if they do not reach a minimum size.

The control module 22 determines image region signal series relating to the image regions based on the signal series 31 of the combined image areas.

Subsequently, the image region signal series are transmitted from the control module 22 to the processing model as signal series 31 in order to determine the respective analyte type or the signal portions of the signal components of the signal series 31 based on the image region signal series.

For example, for each of the analyte types or signal components to be identified, the codebook 23 comprises analyte or signal component contextual information that indicates, for example, a maximum size for an image region depending on the analyte type, that indicates, for example, where in a sample, for example in which of the above-described components of a cell the respective analyte types may occur, or which of the analyte types may be colocalized in the sample at which locations.

The analyte region determination may accordingly take into account this signal component context information and, if necessary, combine or separate analyte regions, determine new analyte region signal series corresponding to the combining or separating, and re-input the newly determined signal series into the processing model to determine the signal portions of the signal components.

The signal component context information further comprises at least one of the following: information about a location of an analyte type in a sample, information about a number of expected analyte types, information about co-localizations of certain analyte types in certain areas of a sample, information about a maximum number of analyte types in certain areas of the sample, a user ID, information about the experiment such as the experiment type or the sample type, and information about a background portion in different areas of the sample.

According to the present invention, the processing model may output as a signal portion, for example, a relative signal portion, an absolute signal portion, or only a binary signal portion. Furthermore, the processing model may also output, as a signal portion, probabilities that the signal component has a signal portion in a signal series 31.

According to the examples described above, the processing model implements the matrix multiplication in the last layer and outputs a sum for each of the signal components in the codebook matrix that indicates how many of the colored signals of the binarized signal series meet true values of the target bit series 35 corresponding to the respective signal component during matrix multiplication. This processing model result output can be interpreted to mean that all signal components for which the sum is greater than a threshold value have a signal portion in the signal series 31. For example, if the number of coloring rounds is 16, as in the examples described above, and an expected number of colored signals for each analyte type is 5, and a relatively good signal-to-noise ratio is expected, then all signal components with a sum of 4 or greater may be interpreted as potential signal components of the binarized signal series.

This threshold can be chosen variably depending on how many colored signals are used to code analyte types, how large the Hamming distance of the target bit series 35 of the different analyte types is, and how many coloring rounds an experiment comprises.

After the processing model has output the signal portions of the various signal components or determined which of the signal components have a signal portion in a signal series 31, the specified signal portions of the signal components in the signal series 31 are checked and verified according to this embodiment in a following step S4.

In step S4, the target bit series 35 of the signal components of the codebook 23 that have a signal portion in the signal series 31 according to the result output of the processing model are first exported from the codebook 23.

The information that certain signal components have a signal portion in a signal series 31 can, for example, simply be a binary vector in which all signal components correspond to one component, which vector has the value “1” for all signal components that may have a signal portion in the signal series 31. The remaining components of the vector that correspond to the signal components that do not have a signal portion in the signal series 31 have the value “0.”

Alternatively, the signal components that have a signal portion in a signal series 31 can also be determined on the basis of the threshold value described above with regard to the examples. The result output for this case is again a vector in which each component corresponds to a signal component, and signal components in which the sum of the matrix multiplication is greater than the threshold value have a signal portion in the signal series 31.

According to the example described above, these are just the signal components whose entries in the result output total greater than 4.

According to a further alternative, the processing model may also have been trained directly to output signal portions of the respective signal components or to output probabilities that a particular signal component contributes to a signal series 31.

After determining the signal components that have a signal portion in the signal series 31, a background signal of the signal series 31 is determined based on the signal series of the surrounding image areas using a background correction method.

The specified background signal is subtracted from the signal series 31 to obtain a background-corrected measurement data vector. The background-corrected measurement data vector comprises 16 entries for an experiment with 16 coloring rounds as in the examples described above.

According to one alternative, another background correction method can also be used, as described further above. A background correction can also be omitted completely, and the background can be used as an independent signal component instead.

After the background correction, the background-corrected measurement data vector is normalized to the length “1” to obtain a normalized background-corrected measurement data vector x.

Using the optimization method, based on the signal portions of the signal component for each pair (T_{A}, T_{B}) of the signal components that may have a signal portion in the signal series 31 according to the result output, the signal portion function is then optimized in such a way that the signal portion function becomes minimal. The signal portion function \hat{α}_{A,B} IS:

\hat{α}_{A,B}=argmin_{α}∥x·(αx_{A}+(1−α)x_{b}∥₂, with a α∈(0,1),

where α is the mixing ratio of the two signal components; since the special case with only two analyte types T_{A} and T_{B} is considered here, this is a one-dimensional optimization problem. In this case, x_{A} is the target bit series 35 of the analyte type T_{A}, x_{B} is the target bit series 35 of the analyte type T_{B}. α is the signal portion of the analyte type T_{A} and (1−α) is the signal portion of the analyte type T_{B}. Below, α is optimized so that the signal portion function \hat{α}_{A,B} becomes minimal.

In a next step, the analyte pair (T_{A}, T_{B}) is then selected for which the signal portion function \hat{α}_{A,B} is minimal, the mixing ratio of the analyte pair can then be determined from the α of the signal portion function α_{A,B}, and the respective signal portions can then be determined from the mixing ratio.

In the optimization procedure described here, various boundary conditions are included in the optimization. On the one hand, the signal portion a is limited to the value range between 0 and 1.

Furthermore, the entries of the target bit series 35 comprise only ones and zeros. Moreover, a linear combination of only two signal components is optimized by means of the signal portion function \hat{α}_{A,B}.

In addition, the optimization of the signal portion function is limited to signal components that have a signal portion in the signal series 31 with a certain probability according to the result output.

According to a variant of the embodiment, the signal portion function can also be set up as linear combinations of, for example, three, four, or more signal components. With suitable boundary conditions and/or regularizations, correspondingly more complex signal portion functions can also be optimized in such a way that the signal portions of the respective signal components can be determined.

According to one alternative in which the processing model outputs the signal portions of the respective signal components of the signal series 31 directly, the signal portions may be included as output values of the signal portions in the linear combination of the signal components in the signal portion function in step S4.

The optimization of the signal portion function on the basis of the signal portions is carried out, for example, by means of a conventional optimization method. According to the present embodiment, the optimization is performed by means of a non-negative matrix factorization, NMF for short.

According to further alternatives, the optimization method can be any classical optimization method, in particular a convex optimization, a non-convex optimization, a concave optimization, a linear optimization, or a non-linear optimization, wherein the classical optimization method is performed with or without constraints, preferably with constraints, in particular boundary conditions.

According to one alternative, the signal portion function can be optimized by using one of the following algorithms: a non-negative matrix factorization, a main component analysis, a discriminant function, or a singular value decomposition.

As further boundary condition or regularizations, signal component context information can also be included in the optimizations, as described above in reference to the processing model.

If the optimization comprises a main component analysis, for example, the transformation matrix of the main component analysis may be a codebook matrix or an analyte signal series matrix. The codebook matrix comprises specifically the vectors of the target bit series 35 as entries. The analyte signal series matrix comprises specifically vectors of the signal series 31 of the different analyte types of the possible signal components as entries.

As described above with regard to the matrix multiplication in an output layer of a processing model, a measure of a portion of the respective signal component in the signal series 31 is obtained by multiplying the signal series 31 by the transformation matrix. Based on this measure, the signal components with the largest portions can then each be specified as a constraint for the optimization in a classical optimization method, for example the two, three, four, or five signal components with the highest portions.

According to a step S5, after the optimization of the signal portion functions and after the determination of the minimum signal portion function, the signal portions are assigned to the respective signal components according to the minimum signal portion function. By first selecting possible signal components by means of a processing model and limiting the optimization of the signal portion function to the specified signal components, considerably fewer computing resources are required to solve the optimization problem.

After the signal portions have been determined as accurately as possible by using the optimization procedure, the annotated data set can be expanded to include signal series 31 comprising mixed or combined signal series or signal portions from a plurality of signal components based on the signal portions specified using the optimization procedure. After having compiled the extended annotated data set, the training of the processing model can be improved with the extended data set.

According to another embodiment, step S4 is performed to optimize the signal portion function without previously inputting the signal series 31 into the processing model.

Accordingly, the signal portion function must be determined for all signal series 31 of all image areas from the image series 19, and, in addition, depending on the number of signal components that are included in the linear combination of the signal portion function, a corresponding number of signal portion functions with a corresponding number of different signal components must be determined in each case for the signal portions of the signal components.

Again, a minimum signal portion function is selected from the plurality of optimized signal portion functions, on the basis of which the signal portions of the plurality of signal components are specified or selected.

A signal portion function can be chosen, for example, that specifies the signal portions by means of a linear combination of two, three, four, or more signal components. Again, suitable boundary conditions or regularizations are used for the optimization.

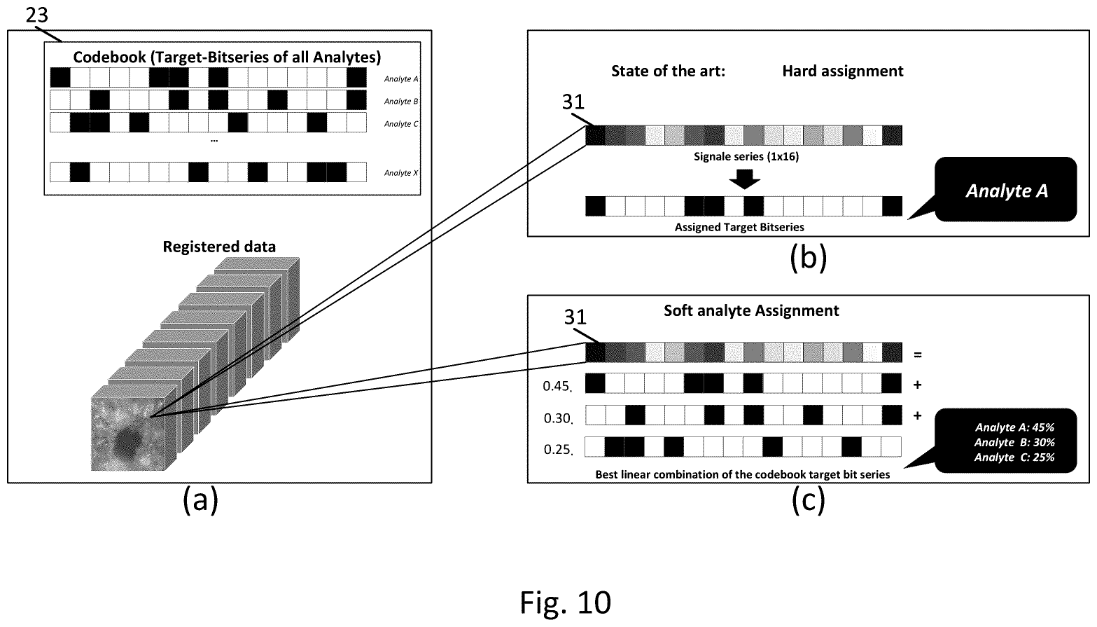

According to one example (see FIG. 10), the signal portion function is a linear combination of three signal components. In each case, the signal series 31 are extracted from the registered data acquired and then the signal series of signal components in the respective signal series 31 are determined by means of an optimization procedure, as described above.

In contrast to the optimization method described above, however, the optimization is performed with all target bit series 35, i.e., all possible signal components of the codebook 23, i.e., all possible linear combinations of the target bit series 35 are optimized in each case and then a minimum signal portion function is selected from the set of the optimized signal portion functions.

According to this embodiment, signal component context information may also be included in the optimization procedure as boundary conditions or regularizations.

One result of the optimization by means of a signal portion function that determines the signal portions of the signal components by means of a linear combination of three signal components is schematically shown in FIG. 10 (c). A result of the optimization then provides the respective portion of the analytes for the minimum signal portion function. The signal series 31 as shown in FIG. 10 is comprised of analytes A, B, and C, as can be seen in FIG. 10 (c). In the prior art, an analyte assignment would indicate that the signal series 31 in question originates from analyte A, as shown in FIG. 10 (b). According to the present invention, however, a signal portion of 45% is assigned to analyte A, a signal portion of 35% is assigned to analyte B, and a signal portion of 20% is assigned to analyte C.

Furthermore, according to one variant, the determination of the signal composition may further comprise a non-maximum suppression.

When the image series 19 is acquired, signals from analytical markers or markers coupled to analytes 39 are mapped onto a plurality of pixels due to the optical properties of the objectives 8 of the microscope 2, in particular the point spread function of the microscope 2. For each of the signal series 31 of the pixels or of the image areas 25 belonging to the same analyte 39, the method described would output a finding of an analyte 39 in the sample depending on the number of pixels onto which an analyte is mapped, i.e., a plurality of the analytes 39 actually present in the sample would thus be found by means of the method described.

By means of non-maximum suppression, the signal series 31 of adjacent image areas 25 are processed or filtered so that only a single signal composition is output for an image section whose area corresponds approximately to the point spread function of the microscope 2.

The non-maximum suppression searches for and filters out of the plurality of specified signal compositions of the plurality of signal series 31 the one whose result corresponds to a maximum score, i.e., whose result corresponds to the correct result with a highest probability. In the case that the processing model outputs probability distributions across the signal components, for example, this can be precisely the result with the highest probabilities. If the signal components were specified by means of a classical optimization algorithm, for example, the result with the smallest error should be selected by non-maximum suppression. Any other soft assignment carried out by means of a processing model can also be evaluated by the non-maximum suppression and a result that is correspondingly evaluated as maximally trustworthy can be selected as the maximum.

In particular, the non-maximum suppression can also be applied to the determination of an image region as described above. For an image region to be determined and the corresponding image region signal series, the signal composition is determined for different compositions of the image region from different image areas 25 based on the respective image region signal series, and a score is determined for the signal composition determined in this way, which reflects how trustworthy the determined signal composition is. Based on the score, the image region and its corresponding image region signal series whose score is currently the maximum is then selected.

It is conceivable, for example, that for signal series 31 of image areas 25 in the center of such an image region, the colored signals can be distinguished particularly well from the uncolored signals, while for image areas 25 at the edge of the image region, it is very difficult to distinguish the colored signals from the uncolored signals. Therefore, image areas 25 located at the edge would potentially degrade a score of a larger image region, which is why an image region is limited to, for example, central pixels or image areas 25 with relatively bright colored signals. The image regions could be determined by means of the non-maximum suppression in such a way that the signal composition of the image region signal series can be determined particularly well.

According to a further embodiment, the evaluation device 4 further comprises a candidate extraction module 27.

The candidate extraction module 27 is designed to extract from the image data of the image series 19 a plurality of signal series 31 of a respective image area 25 of the image series 19 and to filter out candidate signal series from the extracted signal series 31, wherein candidate signal series are signal series 31 of image areas 25 that have a high probability of having captured image signals from analytes 39, i.e., in some of the image areas 25 of the image series 19, the signal series 31 comprise image signals originating from a marker coupled to an analyte 39.

The candidate extraction module 27 is implemented as a neural network called a candidate extraction model that is, for example, trained to detect and output candidate signal series in the extracted signal series.

During the training, the control module 22 reads a portion of the image data of an annotated data set from the memory module 20 and transmits it to the candidate extraction module 27. The control module 22 determines an objective function based on the result outputs of the candidate extraction model and on the target data in the annotated data set and optimizes the objective function by adjusting the model parameters of the candidate extraction model based on the objective function.

The training is carried out, for example, by using a stochastic gradient descent method. Any other training method may be used as well. Once training is complete, the control module 22 stores the model parameters of the candidate extraction model in the memory module 20.

During the inference, the candidate extraction module 27 transmits the candidate signal series output from the candidate extraction model either to the control module 22, which stores the candidate signal series in the memory module 20 for later analysis purposes, or directly to the processing module 24, which then determines the signal composition of the candidate signal series according to the candidate signal series as described above.

Similar to the processing model, the candidate extraction model can be implemented as a neural network, a convolutional neural network (CNN), a multi-layer perceptron (MLP), or as a sequential network, for example a recurrent neural network (RNN) or a transformer network.

The candidate extraction model is also trained in step S2.

According to this embodiment, the candidate extraction model is trained to identify candidate signal series based on a number of colored signals or to identify each of the candidate signal series based on a characteristic signature comprising at least one particular ratio. To distinguish the colored signals from the uncolored signals, the candidate extraction model learns to identify at least one particular ratio of colored signal to uncolored signal, colored signal to colored signal, uncolored signal to colored signal, or uncolored signal to uncolored signal in a candidate signal series. This means that a candidate signal series comprises at least a particular ratio of a colored and/or uncolored signal of the respective signal series 31 to at least one other of the colored and/or uncolored signals of the respective signal series 31.

The particular ratio may be a certain distance or difference between the image signals, a quotient between the image signals, a certain number of image signals with a higher image signal than the others, wherein the ratio may be learned for a normalized image signal or for non-normalized image signals, respectively.

According to this embodiment, the candidate extraction model is a fully convolutional network 37. The candidate extraction model is first trained as a classification model, which is a fully connected network 38 with fully connected layers, using the signal series 31 of individual image areas 25 stored as a training signal series in step S1. For this purpose, the control module 22 inputs signal series 31 from the annotated data set into the candidate extraction model. The classification model assigns a class to the signal series 31 that indicates whether the signal series 31 is a candidate signal series. A candidate signal series is a signal series 31 that either has the characteristic signature or has a high probability of having the characteristic signature, or that has a particular ratio of colored signals or uncolored signals, or a certain number of the colored signals and/or uncolored signals.

The classification model can either be a binary classifier, in which case a “1” indicates a candidate signal series, but the class assignment can also be soft in that the classification model outputs a probability of belonging to the respective class for each class.

The control module 22 in turn controls the training as well as the training of the processing model.

According to one alternative, the candidate extraction model may also be an image-to-image model that learns an image-to-image mapping. A target output in the annotated data set is then either a distance value indicating how far away the respective image area 25 is from a closest image area 25 with a candidate signal series, or a probability value indicating how likely the image area 25 is to capture a candidate image series.

According to another alternative, the candidate extraction model is a detection model. The detection model simply outputs a list of image areas 25 that detect a candidate signal series.

The signal series-agnostic training and the hard-negative mining can also be performed for the candidate extraction model, as described above.

When training the candidate extraction model, a pre-trained model can be selected from a set of pre-trained models and the pre-trained model can be adapted to a new experiment by means of transfer learning.

If the analyte data evaluation system 1 also comprises the candidate extraction model as described herein, the control module 22 inputs the extracted signal series 31 into the candidate extraction model and the candidate signal series identified by the candidate extraction model are then transmitted to the processing model for further analysis.

According to a further alternative, the signal composition for each of the signal series 31 may also comprise a background signal component. For this purpose, image signals are determined from image areas 25 surrounding the image area 25 of the signal series 31. The processing model captures a receptive field, for example, whose outer dimensions correspond to twice the dimensions of an area of a point spread function of the microscope 2, i.e., whose area is four times the area of the point spread function.

The image signals of a marked analyte are, for example, mapped onto an analyte area in an image 5 whose area is equal to the point spread function of the microscope 2. If the receptive field of the processing model captures the signal series 31 of a central image area of the analyte area, the processing model can be trained, for example, to determine the background signal component using image signals from image areas outside the analyte area. The analyte area is determined by using non-maximum suppression, for example. The background signal component determined this way can then be used to perform a background correction.

List of Reference Numerals

| | |
|---|---|
| 1 | Analyte data evaluation system |
| 2 | Microscope |
| 3 | Control device |
| 4 | Evaluation device |
| 5 | Microscope image |
| 6 | Stand |
| 7 | Nosepiece |
| 8 | (Mounted) objective |
| 9 | Specimen stage |
| 10 | Holding frame |
| 11 | Specimen carrier |
| 12 | Microscope camera |
| 13 | Fluorescent illumination device |
| 14 | Transmitted light illumination device |
| 15 | Overview camera |
| 16 | Field of view |
| 17 | Mirror |
| 18 | Screen |
| 19 | Image series |
| 20 | Memory module |
| 21 | Channel |
| 22 | Control module |
| 23 | Codebook |
| 24 | Processing module |
| 25 | Image area |
| 26 | Background image area |
| 27 | Candidate extraction module |
| 31 | Signal series |
| 35 | Target bit series |
| 36 | Segmentation mask |
| 37 | Fully convolutional network |
| 38 | Fully connected network |
| 39 | Analyte |
| 40 | Probability distribution |
| 41 | Binarization |
| 42 | Probability series |

US Patent Application
Carl Zeiss Microscopy GmbH
P345322US

1. Method for training a machine learning system with a processing model, wherein the processing model is trained to determine a signal composition of signal series of image areas of an image series, wherein the signal composition comprises signal portions for different signal components to be identified, the image series is generated by marking analytes with markers in a plurality of coloring rounds and detecting the markers with a camera, the camera acquires an image of the image series in each coloring round, the markers are selected in such a manner that signal series of analytes in an image area across the image series comprise colored signals and uncolored signals, the colored and uncolored signals of the signal series of analytes have at least one particular ratio of one of the colored and/or uncolored signals of the respective signal series to at least one other of the colored and/or uncolored signals of the respective signal series, and/or the signal series of analytes have a characteristic signature comprising the at least one particular ratio, comprising:
providing an annotated data set, wherein the annotated data set comprises input signal series for the signal components as well as corresponding target outputs, the signal components comprising at least one signal component for each analyte type to be identified, and signal series of analytes comprising a specific sequence of the colored and uncolored signals, based on which an analyte type can be assigned to the signal series, and
optimizing an objective function by adjusting the model parameters of the processing model, wherein the objective function is calculated based on a result output from the processing model and the target output.

2. Method according to claim 1, wherein the annotated data set further comprises input signal series from background image areas, wherein the background image areas are image areas of the image series in which no signals from analytes are captured, and the target output for background image areas forms at least one distinct signal component in the set of signal components.

3. Method according to claim 1, wherein the processing model is a classification model, the result output indicates the signal portion of the signal components of the input signal series, or the result output is a probability distribution, each indicating the probability of belonging to one of the signal components, and the objective function detects a difference between the result output and the target output.

4. Method according to claim 1, wherein an objective function is optimized in a plurality of rounds, and, in some of the rounds, the sequence of the colored and uncolored signals of one of the input signal series is changed such that the changed sequence corresponds to a sequence of one other of the analyte types to be identified, and the target output corresponding to the changed sequence is used accordingly for the optimization of the objective function.

5. Method according to claim 1, wherein the objective function is a classification loss and the result output for each entry has a value between 0 and 1 indicating a probability that the respective signal series comprises signal portions of the respective signal component.

6. Method according to claim 1, wherein the target output is a target bit series, and the target output comprises a true bit for each colored signal in the input signal series and a false bit for each uncolored signal.

7. Method according to the preceding claim 6, wherein the target output for signal series of background image areas comprises only false values.

8. Method according to claim 6, wherein the result output is a result bit series, wherein the processing model is trained to assign a true bit to each colored signal in the input signal series and a false bit to each uncolored signal in the input signal series, and the objective function detects a difference between the result bit series and the target output.

9. Method according to claim 6, wherein the result output is a probability distribution in which each image signal of the input signal series is associated with a probability that the image signal is a colored signal or not, and the objective function detects a difference between the result output and the target output.

10. Method according to claim 6, wherein the result output for each of the entries has a value between 0 and 1 indicating a probability as to whether or not a colored signal has been captured.

11. Method according to claim 1, wherein the processing model is a fully convolutional network, which is either trained directly as a fully convolutional network or has been trained as a classification model with fully connected layers with signal series of individual image areas and after the training, the classification model is transformed, through replacement of the fully connected layers with convolutional layers, into the fully convolutional network that can process the signal series of all image areas of the image series simultaneously.

12. Method according to claim 2, wherein a calculation of the objective function comprises:
calculating a candidate group of candidate objective functions for each input signal series of analytes, wherein, for each of the candidate objective functions, a different one of the colored signals of the input signal series is disregarded in the calculation of the candidate objective function, for example by being set to zero or replaced by an uncolored signal, and/or one or more image signals of the input signal series for input signal series of a background image area for each of the candidate objective functions are not taken into account in the calculation of the candidate objective functions by omitting the corresponding colored signals from the calculation or replacing them with uncolored signals, and
selecting an objective function of choice from the candidate group, wherein the objective function of choice is the candidate objective function of the candidate group that has either a second largest or a third largest or a fourth largest difference between the target bit series and the result bit series.

13. Method according to claim 6, wherein the processing model is an embedding model that determines an embedding in an embedding space for the embedding inputs, the embedding inputs comprise the input signal series and the target outputs, the result outputs comprise the embeddings of the input signal series, the target embeddings comprise the embeddings of the target outputs, and the optimization of the objective functions simultaneously minimizes the difference between the embeddings of the embedding inputs of the same signal component and maximizes the difference between the embeddings based on the embedding inputs of different signal components.

14. Method according to claim 6, wherein an objective function is optimized in a plurality of rounds, and, in some of the rounds, comprises a randomization of the input signal series, wherein the randomization comprises one or more of the following:
swapping a sequence of the image signals of the input signal series and correspondingly swapping the corresponding entries of the target output, and
randomly selecting a first number of colored signals and a second number of uncolored signals from the set of input signal series and producing the respectively corresponding target output.

15. Method according to claim 1, wherein an objective function is optimized in a plurality of rounds and, in some of the rounds, comprises an augmentation of the input signal series, wherein the augmentation comprises one or more of the following:
replacing a single one of the colored signals of the input signal series with an uncolored signal, wherein the uncolored signal is generated either by lowering the colored signal or by replacing the colored signal with an image signal from the vicinity of the image area of the input signal series from another coloring round or from another location in the sample,
randomly adding noise to some of the image signals of the input image series, for example the image signals of an input signal series, one of the images of the image series, or all images of the image series,
shifting and/or rotating the images of the image series with respect to each other, for example by less than two pixels or less than or equal to one pixel, for example half a pixel,
replacing a single one of the uncolored signals of the input signal series with a colored signal,
shifting the image signals of at least one of the images of the image series by a constant value,
generating combined signal series by linearly combining a plurality of the signal series of different analyte types, wherein each of the analyte types is included in the sum with an analyte weight and the objective function preferably also detects a difference between the analyte weights and a specific signal portion of the respective signal component of the signal composition, and
shifting the image signals of the input signal series by a constant value.

16. Method according to claim 1, wherein the input signal series is transformed into a transformed input signal series by means of a transformation, and the transformed input signal series is input into the processing model, wherein the transformation comprises in particular one or more of the following:
a main component analysis,
a main axis transformation,
a singular value decomposition,
a normalization, wherein the normalization comprises a normalizing of the image signals across an image or a normalizing of the image signals across a signal series, or both.

17. Method according to claim 1, wherein the annotated data set is generated with at least one of the following steps:
simulating signals of the different markers by using a representative background image and a known point spread function of the microscope,
generating the annotated data set by means of a generative model trained on comparable data,
acquiring reference images comprising at least one background image and, for each of the background images for each of the analyte types, at least one image in which analytes of the respective analyte types are marked,
performing a classical method for the spatial identification of analytes,
acquiring a representative background image and subtracting the image signals of the representative background image pixel by pixel from the image signals of the image series on which the annotated data set is based, prior to providing the annotated data set, so that the annotated data set comprises only background-corrected signal series.

18. Method for determining a signal composition of signal series of an image series with an analyte data evaluation system, wherein the image series is generated by marking analytes with markers in a plurality of coloring rounds and detecting the markers with a camera, wherein the camera acquires an image of the image series in each coloring round, the markers are selected in such a way that the signal series of analytes in an image area across the image series comprise colored and uncolored signals and the signal series of different analyte types each have a specific sequence of colored signals and uncolored signals, and the different analyte types can be identified from the specific sequences, comprising:
receiving signal series,
importing a codebook, wherein the codebook comprises a target series for all signal components, the target series comprise analyte target series, and the analyte target series comprise a sequence of true and false values according to the specific sequences of the signal series of the different analyte types, and
determining the signal composition for each of the signal series, wherein the signal components are assigned a signal portion in the respective signal series according to the signal composition.

19. Method according to claim 18, wherein the signal composition is determined on the basis of a signal portion function, wherein the signal portion function detects a difference between the signal series and a linear combination of a plurality of the target series, and the signal composition determination further comprises:
optimizing the signal portion function based on the signal portions.

20. Method according to claim 18, wherein the signal portion function is optimized by using at least one of the following algorithms:
a classical optimization algorithm,
a non-negative matrix factorization,
a main component analysis,
a discriminant function, or
a singular value decomposition.

21. Method according to claim 18, wherein the optimization is performed based on predetermined constraints.

22. Method according to the preceding claim 21, wherein the constraints comprise at least one of the following:
the values of the signal portions can be non-negative,
the entries in the target series can be non-negative,
the number of the colored signals in a target series is predetermined for all analyte types in the codebook, for example as a fixed value or as an interval,
the number of the colored signals is specified individually for each of the nominal sequences.

23. Method according to the preceding claim 22, wherein the optimization is performed with regularizations.

24. Method according to the preceding claim 23, wherein the regularizations comprise at least one of the following:
a predetermined maximum number of distinct signal components,
an expected number of analyte types,
a limitation of the combinability of the analyte types with each other,
a limitation of the optimization to sparse solutions.

25. Method according to claim 18, wherein the determination of a signal composition comprises:
inputting the signal series into a processing model, wherein the processing model was trained to provide a result output from which the signal portion to the respective signal series is determined for each signal component.

26. Method according to the preceding claim 25, wherein the processing model is a classification model, the result output for each signal series is a probability distribution across the signal components to be identified, each indicating a probability of belonging to one of the signal components to be identified, and the signal portion is determined on the basis of the probability distribution.

27. Method according to the preceding claim 25, wherein the result output is based on a multiplication of a layer output of the processing model by an analyte matrix, wherein the analyte matrix is based on the target series of the codebook, and the result output provides a value for each of the signal components from which the signal portion is determined.

28. Method according to claim 27, wherein the processing model is a classification model, wherein the layer output comprises a probability distribution assigning a probability of being a colored signal to each image signal of a signal series, the target series are bit series, comprising a true value for each expected colored signal and a false value for each expected uncolored signal, and the result output for each signal series comprises a sum of the probability values of the layer output corresponding to a true value of the target series, and the signal portion is determined on the basis of the sum.

29. Method according to claim 27, wherein the processing model is an embedding model that determines the respective embeddings of the signal series and target series in an embedding space such that the layer output is a result embedding and the analyte matrix is based on the embeddings of the target series, wherein the embedding model has been trained to map signal series of a particular analyte type and their corresponding target series to the embedding space such that the different embeddings that correspond to the same signal component have the smallest possible spacing in the embedding space, the embeddings that correspond to different signal components have the greatest possible spacing, and the embeddings of signal series with signal portions of a plurality of signal components have the smallest possible spacing from the embeddings of the respective plurality of signal components and the greatest possible spacing from the embeddings of the remaining signal components.

30. Method according to claim 25, wherein, during the training of the processing model, an annotated data set was used that comprises training signal series and the corresponding target series for a plurality of analyte types to be identified, and, during the training, training signal series and the corresponding target series of different analyte types are linearly combined in order to train the processing model on mixed signal series as well.

31. Method according to claim 19, wherein the determination of a signal composition comprises:
clustering the extracted signal series by means of a cluster analysis algorithm, wherein a number of predetermined clusters is at least equal to a number of the signal components,
determining a cluster center for each of the clusters,
determining at least one target cluster center for each of the signal components on the basis of the target series,
determining the cluster distances of the cluster center to the target cluster centers for each of the cluster centers, and
assigning the clusters to one of the signal components on the basis of the cluster distances,
determining the distance to the respective cluster centers for each of the signal series, and
determining the signal portion based on the distances.

32. Method according to claim 1, wherein n of the coloring rounds each correspond to a marking round and each analyte type is detected in only one of the n coloring rounds of a marking round, wherein the n markers are embodied in such a way that only one of the n markers is coupled to each of the analyte types in each marking round and each of the n markers is acquired in a different color contrast and, for example, when determining the signal composition, it is taken into account as a boundary condition that an analyte is marked with a marker in only one of the n coloring rounds of a marking round.

33. Method according to claim 32, wherein a total of n*m coloring rounds are carried out and correspondingly n*m images are acquired and a signal series comprises n*m image signals, wherein each analyte type has a colored signal in at the most m of the coloring rounds and, for example, when determining the signal composition, it is taken into account as a boundary condition that an analyte is marked with a marker in only at most m of the coloring rounds.

34. Method according to claim 1, wherein signal component context information is included in the determination of a signal composition, wherein the signal component context information comprises at least one of the following:
information about a location of an analyte type in a sample,
information about a number of expected analyte types,
information about the co-localizations of certain analyte types in certain areas in a sample,
information about a maximum number of analyte types in certain areas of the sample
information about a background portion in different areas of the sample.

35. Method according to claim 1, wherein the method further comprises a step of performing a background correction of the image signals of the image series prior to the determination of a signal composition, wherein performing the background correction comprises one or more of the following:
a rolling-ball method,
a filtering such as a top-hat method, a homomorphic filtering, a low-pass filtering, wherein the result of the low-pass filtering is subtracted from the signal, or a temporal filtering,
a background correction by means of an image-to-image model,
a background correction by means of mixed models,
a background correction by means of a mean-shift method,
a background correction by means of a main component analysis,
a background correction by means of a non-negative matrix factorization, a background correction by means of an excitation of the auto-fluorescence by means of a specific laser for all image areas of the image series, wherein the specific laser corresponds to an excitation spectral range of one of the markers used and the analytes are not marked with markers, or
a background correction by means of an excitation of the auto-fluorescence by means of a non-specific laser for all image areas of the image series.

36. Method according to claim 1, wherein, when determining a signal composition for each of the signal series, a background component is also included as another one of the signal components with another signal portion.

37. Method according to claim 1, wherein, when determining a signal composition for each of the signal series, a noise component is also included as another one of the signal components with another signal portion.

38. Method according to claim 1, wherein the method further comprises a normalization of the image signals, wherein the normalization comprises at least one of the following:
normalizing the image signals across an entire image,
normalizing the image signals across all images of the image series,
normalizing the image signals across a signal series,
normalizing the image signals across a signal series such that relative signal portions are determined, or
normalizing the image signals based on a color contrast of the image signals.

39. Method according to claim 1, wherein the image areas each comprise, for example, only one pixel, an area of contiguous pixels, or a contiguous volume in an image stack, and, for example, the signal series is a tensor comprising entries for each of the pixels in the image area and each of the coloring rounds, or values of adjacent pixels are included in the tensor as combined entries.

40. Method according to claim 1, further comprising a determination of an image region, the determination of an image region comprising:
combining adjacent image areas into an image region, if the adjacent image areas comprise signal series with the same signal components, wherein the combining of adjacent image areas comprises, for example, non-maximum suppression.

41. Method according to the preceding claim 40, wherein the determination of an image region furthermore comprises: verifying the image regions, wherein verifying an image region comprises at least one of the following:
separating the image region into two or more image regions if the image region exceeds a maximum size,
separating the image region into two or more image regions if the image regions are each connected only by a few bridge pixels and/or it is apparent from a shape of the image region that two image regions intersect here,
separating the image region based on signal component context information, wherein the signal component context information comprises, for example:
information about a size of an image region depending on the analyte type, information about a location of an image region in a sample, information about co-localizations of certain analyte types in certain areas or in a location in a sample, the expected analyte densities depending on a location of the image region in a sample;
discarding image regions if an image region falls below a minimum size or has a shape that cannot be reliably assigned to an analyte type.

42. Method according to the preceding claim 41, wherein the maximum size of the image region is selected depending on the point spread function of an imaging device.

43. Method according to the preceding claim 42, wherein the determination of an image region furthermore comprises:
determining an image region signal series based on the signal series of the image areas that the image region is comprised of, and
the determination of the signal composition is carried out based on the image region signal series and comprises a combining of image signals from adjacent image areas into a combined image signal of the image region.

44. Method according to claim 40, wherein the determination of an image region is carried out after a signal composition has been determined for each of the signal series.

45. Method according to claim 18, wherein the determination of a signal composition comprises non-maximum suppression.

46. Method according to claim 1, wherein the signal portion indicates a relative portion of the image signal of the respective signal component in the image signals of the signal series.

47. Method according to claim 1, wherein the signal portion indicates an absolute portion of the image signal of the respective signal component in the image signals of the signal series.

48. Method according to claim 29, wherein, after the determination of the signal composition, the determined signal portions are used as output values for optimizing a signal portion function, and subsequently, using the output values, the optimization of the signal portion function determines the signal portions once again by means of a method for determining a signal composition of signal series of an image series with an analyte data evaluation system, wherein the image series is generated by marking analytes with markers in a plurality of coloring rounds and detecting the markers with a camera, wherein the camera acquires an image of the image series in each coloring round, the markers are selected in such a way that the signal series of analytes in an image area across the image series comprise colored and uncolored signals and the signal series of different analyte types each have a specific sequence of colored signals and uncolored signals, and the different analyte types can be identified from the specific sequences, comprising:
receiving signal series,
importing a codebook, wherein the codebook comprises a target series for all signal components, the target series comprise analyte target series, and the analyte target series comprise a sequence of true and false values according to the specific sequences of the signal series of the different analyte types, and
determining the signal composition for each of the signal series, wherein the signal components are assigned a signal portion in the respective signal series according to the signal composition.

49. Method according to claim 29, further comprising:
generating an extended annotated data set based on the specified signal portions, wherein the signal portions are verified before being included in the extended annotated data set, in particular by determining the specified signal portions of the signal series again with a plurality of signal components and including them in the extended data set if the determined signal portions match, and
performing the method for training a machine learning system with at least the extended annotated data set as the annotated data set by employing a method for training a machine learning system with a processing model, wherein the processing model is trained to determine a signal composition of signal series of image areas of an image series, wherein the signal composition comprises signal portions for different signal components to be identified, the image series is generated by marking analytes with markers in a plurality of coloring rounds and detecting the markers with a camera, the camera acquires an image of the image series in each coloring round, the markers are selected in such a manner that signal series of analytes in an image area across the image series comprise colored signals and uncolored signals, the colored and uncolored signals of the signal series of analytes have at least one particular ratio of one of the colored and/or uncolored signals of the respective signal series to at least one other of the colored and/or uncolored signals of the respective signal series, and/or the signal series of analytes have a characteristic signature comprising the at least one particular ratio, comprising:
providing an annotated data set, wherein the annotated data set comprises input signal series for the signal components as well as corresponding target outputs, the signal components comprising at least one signal component for each analyte type to be identified, and signal series of analytes comprising a specific sequence of the colored and uncolored signals, based on which an analyte type can be assigned to the signal series, and
optimizing an objective function by adjusting the model parameters of the processing model, wherein the objective function is calculated based on a result output from the processing model and the target output.

50. Method according to claim 18, wherein the receiving of signal series comprises at least one of the following:
extracting all image areas of the image series,
extracting a random selection of the image areas of the image series,
extracting a selection of the image areas of the image series weighted with a structural property of the image areas, for example, with a higher probability for cells, cell nuclei, and bright pixels,
extracting image areas exclusively from image areas with a minimum degree of image sharpness, and
skipping image areas in which no analytes are to be expected.

51. Method according to claim 1, wherein the image series additionally comprises context information used during the method, wherein the context information comprises, for example:
a type of sample mapped in the microscope images,
a type of specimen carrier used for acquiring the specimen image, for example information as to whether a chamber slide, a microtiter plate, a slide with a coverslip, or a Petri dish was used,
image acquisition parameters, such as information about illumination intensity, exposure duration, filter settings, fluorescence excitation, contrast method, or specimen stage settings,
information about objects in the respective microscope image,
application information indicating the type of application for which the microscope images were acquired,
information about the user who acquired the images.

52. Method according to claim 51, wherein the processing model is selected from a set of processing models, wherein the processing model is selected, for example, manually, automatically, based on context information, or based on a sample type, an experiment type, or a user ID.

53. Method according to claim 25, wherein the processing model is selected from a set of pre-trained processing models, wherein the selection is made, for example, on the basis of context information, is made automatically or is selected by a user, and the set of processing models is preferably kept available locally at a user's facility, is based on a model catalog from the manufacturer, or is kept available online by the manufacturer, and the processing model has been trained in particular according to method 1.

54. Evaluation device for evaluating images of an image series, which is designed in particular as an analyte data evaluation system, comprising the means for carrying out the method according to claim 1.

55. Image processing system comprising an evaluation device according to the preceding claim 54, in particular comprising an image generation device such as a microscope.

56. Computer program product comprising instructions which, when the program is executed by a computer, cause the computer to execute the method according to claim 1, the computer program product being in particular a computer-readable storage medium.

57. Analyte data evaluation system comprising an evaluation device, wherein the evaluation device comprises a processing model that has been trained using the method according to claim 1, in particular comprising an image generation device such as a microscope.
